(19) United States
(12) Patent Application Publication
(10) Pub. No.: US 2025/0188486 A1
(43) Pub. Date: Jun. 12, 2025

(54) NEW GENE RESPONSIBLE FOR CYTOPLASMIC MALE STERILITY

(71) Applicant: VILMORIN & CIE, Paris (FR)

(72) Inventors: Ian SMALL, Wattle Grove (AU); Joanna MELONEK, Crawley (AU)

(73) Assignee: VILMORIN & CIE, Paris (FR)

(21) Appl. No.: 19/034,234

(22) Filed: Jan. 22, 2025

Related U.S. Application Data
(62) Division of application No. 17/429,176, filed on Aug. 6, 2021, now Pat. No. 12,241,076.

(30) Foreign Application Priority Data
Feb. 6, 2019 (EP) 19305141.4

(51) Int. Cl. C12N 15/82; C07K 14/415; C12Q 1/6895

(52) U.S. Cl. CPC C12N 15/8289; C07K 14/415; C12Q 1/6895; C12Q 2600/13; C12Q 2600/158

(57) ABSTRACT

An isolated nucleic acid encoding Orf279 protein of amino acid sequence at least 95% identical to SEQ ID NO: 4. Methods for detecting orf279 DNA, orf279 RNA or Orf279 protein and method for identifying functional Rf gene encoding a protein able to bind to orf279 RNA.

### CROSS-REFERENCE TO RELATED APPLICATIONS

This is a Division of application Ser. No. 17/429,176 filed Aug. 6, 2021, which in turn is a national phase of PCT/EP2020/053036 filed Feb. 6, 2020, which claims the benefit of EP 19305141.4 filed Feb. 6, 2019. The disclosure of the prior applications is hereby incorporated by reference herein in its entirety.

### REFERENCE TO AN ELECTRONIC SEQUENCE LISTING

The present application contains a Sequence Listing that has been submitted electronically and is hereby incorporated by reference in its entirety. The electronic Sequence Listing is named 19720601SubstituteSequenceListing.xml, was created on Mar. 5, 2025, and is 116,615 bytes in size.

### BACKGROUND

By 2050 the world's human population is projected to exceed nine billion (United Nations, 2017). At the same time the area of arable land is predicted to shrink from 0.38 ha per person in 1970 to 0.15 ha per person in 2050 (FAOSTAT 2017). Thus, in order to meet future food demands, the yield per hectare needs to increase, ideally without elevated usage of water or fertilizer. By contributing 11% to the total crop production, wheat is one of the most important small grain crops cultivated worldwide (FAOSTAT 2017, Langridge 2017). After the introduction of Green Revolution crops in 1960, the creation of hybrid varieties in crops such as rice, corn and sorghum lifted their productivity significantly and contributed greatly to overall cereal seed production that today makes up 50% of global food production (FAOSTAT 2017). Due to lack of an efficient pollination control system, hybrid production in wheat relies mainly on the use of chemical hybridizing agents (CHA) and is only marginally applied (Whitford et al., 2013). Likely as a result of this, the rate of wheat yield gains has been slower over the last decade compared to corn or rice yield gains (FAOSTAT 2017, Whitford et al., 2013). The main goal of hybrid production is to take advantage of heterosis to produce more resource-and energy-efficient plants with higher and more stable yields. It is estimated that yield improvements associated with heterosis in wheat could reach up to 15% (Longin et al., 2012). Hybrid production requires a method to block self-pollination of autogamous plants, like wheat, where manual separation of male and female sexual organs is labour-intensive and thus inapplicable on an industrial scale (Chase et al., 2007). A system that has been successfully used for production of hybrids in several crop plants including maize, rice and sorghum is the three-line breeding system based on cytoplasmic male sterility (CMS), a genetically conditioned trait that leads to plant sterility (Chen and Liu 2014; Bohra et al., 2016; Yamagishi and Bhat 2014; Saxena et al., 2015). It requires three types of breeding lines: a cytoplasmic male sterile line (A line), which carries the CMS-causing gene, a maintainer line (B line) which is required for propagating the A line whilst maintaining the sterility and a restorer line (R line) which carries a restorer-of-fertility (Rf) gene able to restore fertility of F1 plants (Chen and Liu 2014). Historically, the strong inbreeding nature governed by the unique architecture and development of wheat flowers complemented by the lack of suitable Rf genes are the major factors limiting the application of CMS to hybrid seed production in wheat (Whitford et al., 2013).

CMS in cultivated wheats originates from interspecific crosses between bread wheat (Triticum aestivum) as a pollen donor and wild wheat e.g. Triticum timopheevii. or related species such as Aegilops or Hordeum, and backcrossing to bread wheat (Whitford et al., 2013). The first case of male sterility in wheat was reported in 1951 when the nucleus of Aegilops caudata cells was substituted by the nucleus from Triticum aestivum (Kihara, 1951). Subsequently the T-type CMS wheat (also known as G-type CMS) was derived from cross between Triticum timopheevii as female parent and bread wheat as the male parent (Wilson and Ross 1962).

It was proposed that the T-CMS is caused by a single gene designated orf256 in the mitochondrial genome of T. timopheevii (Rathburn and Hedgcoth, 1991). Sequence analysis has revealed that the region from −228 to +33 (relative to the start codon) of orf256 is identical to the analogous region from cox1 (encoding subunit 1 of mitochondrial complex IV) in T. aestivum, whereas the rest of orf256, including the 3′ flanking region, is not related to coxl (Rathburn and Hedgcoth, 1991; Song and Hedgcoth, 1994A). Most probably, a single recombination event has led to the formation of orf256 in T. timopheevii mitochondrial DNA (mtDNA) (Rathburn and Hedgcoth, 1991; Song and Hedgcoth, 1994A). It has been documented that although the gene organization of orf256 fragments from T. timopheevi, CMS (T. aestivum nucleus, T. timopheevii mitochondria), and fertility-restored lines are identical, orf256 transcript processing is altered by different nuclear backgrounds (Rathburn and Hedgcoth 1991; Song and Hedgcoth, 1994A). The name Orf256 originates from the 256 amino acids that are encoded by the orf256 coding sequence (Rathburn and Hedgcoth, 1991). Antibodies directed towards a peptide corresponding to a part of the encoded amino acid sequence of orf256 detect a 7 kDa protein on western blots of mitochondrial proteins from T-CMS wheat but not on blots of mitochondrial proteins from T. aestivum, T. timopheevi, or T-CMS plants restored to fertility by introduction of nuclear genes for fertility restoration (Song and Hedgcoth 1994B). Moreover, it has been shown that Orf256 is anchored in the inner mitochondrial membrane (Song and Hedgcoth 1994B). Since the first molecular studies on T-CMS performed by Hedgcoth and colleagues, no follow-up studies were initiated for the next 25 years.

It is known today that restorer of fertility (Rf) proteins are encoded in the nucleus and post-translationally targeted to mitochondria, where they prevent the accumulation of RNA encoding CMS-specific ORFs (Kazama et al., 2008; Bentolia et al., 2002). The majority of Rf genes in higher plants identified to date encode pentatricopeptide repeat (PPR) proteins (Kotchoni et al., 2010; Chen and Liu 2013). The PPR family has highly expanded in land plants, and the members of the family involved in CMS, referred to as Restorer-of-fertility-like (Rfl) tend to exist as gene clusters at two to three genomic positions (Schmitz-Linneweber and Small, 2008, Fujii et al., 2011; Melonek et al, 2016). For example, several Rf genes map to a gene cluster on chromosome 10 in rice, including Rf1a and Rf1b for CMS-Chinsurash Boro II and Rf4 for CMS-wild abortive (Akagi et al., 2004; Wang et al., 2004; Zhang et al., 2002). Recent global analysis of the PPR family in the wheat RefSeq v1.0 genome revealed the presence of 207 Rfl genes, the majority of which are organised in clusters on chromosome 1, 2 and 6 (The International Wheat Genome Sequencing Consortium, 2018).

Several restorer genes controlling fertility in the T-CMS system have been reported in Triticum aestivum, namely Rf1 (chr1A) (Du et al. 1991), Rf2 (chr7D) (Bahl and Maan 1972; Maan et al. 1984), Rf3 (chr1B) (Tahir and Tsunewak.K 1969), Rf4 (chr6B) (Maan et al. 1984), Rf5 (chr6D) (Bahl and Maan 1972), Rf6 (chr5D) (Bahl and Maan 1972), Rf7 (chr7B) (Bahl and Maan 1972) and Rf8 (chr2D) (Sinha et al. 2013). The Rf1 locus was first described in the T. timopheevii introgression line R3 (Livers 1964; Bahl and Maan 1973) and was later found to be located on chromosome 1A (Robertson and Curtis 1967). A restorer locus on this chromosome was also found in three other T. timopheevil introgression lines, the spring wheat accession R113 and its descendants (Bahl and Maan 1973; Maan et al. 1984; Maan 1985; Du et al. 1991). Recently, the Rf1 locus was genetically mapped to a 8.17 Mbp region on chromosome 1A (Greyer et al., 2017). In addition, a modifier locus for Rf1 was identified on chromosome 1B (Greyer et al., 2017). Rf3 was reported as one of the most effective restorer loci (Ma and Sorrells, 1995; Kojima et al, 1997; Ahmed et al 2001; Geyer et al 2016). Two SNP markers allowed the location of the Rf3 locus within a 2 cM fragment on chromosome 1B (Geyer et al, 2017). The genomic location of both Rf1 and Rf3 overlaps with the location of RFL clusters described to be present on the wheat chromosome 1A and 1B (International Wheat Genome Sequencing Consortium, 2018).

For effective use of CMS and Rf genes in hybrid breeding programs, it is crucial to understand the molecular mechanisms linking them in plant mitochondria. The present invention deals with the identification of a new gene, orf279 that causes male sterility in T-CMS wheat, through molecular characterization of the Rf1- and Rf3-associated restoration mechanism. The present invention deals with new molecular and breeding tools developed from orf279 gene sequence information.

### SUMMARY

A first aspect of the invention is an isolated nucleic acid encoding Orf279 protein of amino acid sequence at least 95% identical to SEQ ID NO: 4.

In a second aspect, the present application concerns methods for detecting orf279 DNA, orf279 RNA or Orf279 protein in a sample.

In a third aspect, the present invention relates to a method for identifying a functional Rf gene encoding a protein able to bind to orf279 RNA.

In a fourth aspect, the present invention concerns a method for the design and the optimization of a synthetic PPR protein capable of binding and preventing expression of orf279 RNA.

In a fifth aspect, the present invention relates to a method for obtaining a sterile plant.

In a sixth aspect, the present invention relates to a method for obtaining a fertile wheat plant.

### FIGURES

FIGS. 1A-1D. Expression of orf256 is unaltered by Rf1 and Rf3. FIG. 1A. Schematic overview of the orf256 genomic structure. Binding sites of primers P1-P3 used in the RT-PCR analysis are indicated. FIG. 1B. RT-PCR analysis of the expression of orf256 in different wheat genotypes. M-100 bp ladder, c-water control. Actin was used as an internal reference control. FIG. 1C. Survey of several T-CMS accessions in regard to orf256 processing by Northern blot including six wheat varieties. The WORF256 probe used to detect orf256 was prepared as described previously (Song et al., 1994). FIG. 1D. Mapping of the 5′-ends of orf256 RNA species by 5′-RACE approach. GSP2—Gene specific primer 2. M-100 bp ladder.

FIGS. 2A-2C. Identification of orf279 as a novel RNA associated with T-CMS in wheat. FIGS. 2A-B. Ratio of strand-specific RNA-seq coverage from sterile and restored (Rf1) samples plotted across the mitochondrial genome. RNA-seq coverage of orf279 (FIG. 2B) is much higher in sterile plants whereas coverage of orf256 (FIG. 2A) is similar in both. FIG. 2C. Normalised RNA-seq coverage in the orf279 region. orf279 is indicated by the boxes below the chart, distinguishing the part of orf279 that is identical to atp8 and the orf279-unique region. The number of RNA-seq reads mapped to the central region of orf279 in Rf1 transformants is much lower than in BGA CMS*Fielder. The sharp transition from low to high coverage indicates the probable site of RNA cleavage induced by Rf1.

FIGS. 3A-3B. Orf279 as genetic basis of cytoplasmic male sterility in wheat T-CMS plants. FIG. 3A. Schematic drawing of orf279. The first 97 amino acid residues at the N-terminus of Orf279 correspond to ATP synthase subunit 8 encoded by the mitochondrial atp8 gene. FIG. 3B. 5′-RACE analysis of orf279 transcripts. Arrows indicating Rf1 and Rf3 specific cleavage products, respectively, are shown. The binding site of GSP1—Gene specific primer 1 is indicated in panel A. M-100 bp ladder.

FIGS. 4A-4B: Design of the Synthetic Restorer for orf279 (SRorf279) and Synthetic Restorer for orf256 (SRorf256). Amino acids at position 5 and 35 of each PPR motif were extracted and aligned with an RNA base predicted following the “PPR code” (Barkan et al., 2012). Amino-acid modified during optimization of SRorf279 and SRorf256 proteins are indicated.

FIG. 5: Location of PCR primers on orf279 sequence: primer PP_03004_ORF279-amont-cliv_3_F (SEQ ID NO:6), primer PP_03004_ORF279-amont-cliv_3_R (SEQ ID NO:7), primer PP_03006_ORF279-aval-cliv_4_F (SEQ ID NO:8), primer PP_03006_ORF279-aval-cliv_4_R (SEQ ID NO:9), primer PP_03007_ORF279-cliv_F (SEQ ID NO:10), SEQ ID NO:10 (SEQ ID NO:11), primer PP_03003_atp8_4_F (SEQ ID NO:12), primer PP_03003_atp8_4_R (SEQ ID NO:13).

FIG. 6: Design of two optimized RFL29a proteins. Amino acids at position 5 and 35 of each PPR motif were extracted and aligned with an RNA base predicted following the “PPR code” (Barkan et al., 2012). Amino acids modified during optimization of RFL29a protein are indicated.

FIG. 7: PCR amplification results. Right cluster: amplification of the normal cytoplasm with oligonucleotide AS2 in fertile plants. Left cluster: amplification of the orf279 with oligonucleotide AS1 in sterile plants.

### DETAILED DESCRIPTION

### Definitions

Whenever reference to a “plant” or “plants” is made, it is understood that also plant parts (cells, tissues or organs, seed pods, seeds, severed parts such as roots, leaves, flowers, pollen, etc.), progeny of the plants which retain the distinguishing characteristics of the parents (especially, male fertility associated with the claimed Rf nucleic acids), such as seed obtained by selfing or crossing, e.g. hybrid seeds (obtained by crossing two inbred parent plants), hybrid plants and plant parts derived therefrom are encompassed herein, unless otherwise indicated.

As used herein, the term “wheat plant” refers to species of the genus Triticum as for example, T. aestivumT, T. aethiopicum, T. araraticum, T. boeoticum, T. carthlicum, T. compactum, T. dicoccoides, T. dicoccon, T. durum, T. ispahanicum, T. karamyschevii, T. macha, T. militinae, T. monococcum, T. polonicum, T. spelta, T. sphaerococcum, T. timopheevii, T. turanicum, T. turgidum, T. urartu, T. vavilovii, T. zhukovskyi Faegi. Wheat plant also refers to species of the genera Aegilops and Triticale.

As used herein, the term “restorer of fertility of T. timopheevii CMS cytoplasm” refers to a protein whose expression in a wheat plant containing T. timopheevii CMS cytoplasm contributes to the restoration of the production of pollen.

### New Gene Responsible for CMS

The inventors have discovered that the orf279 gene is responsible for CMS in wheat plants containing T. timopheevii cytoplasm. For clarity, this cytoplasm is herein referred to as T-CMS. It refers to any cytoplasm expressing orf279, a representative cytoplasm is the cytoplasm of T. timopheevii. For example, T-CMS can be present in wheat plant derived from T. timopheevil. T-CMS can also be a wheat plant from which T. timopheevii is derived, like T. araraticum the cultivated form of T. timopheevii.

The invention relates to the isolated nucleic acid encoding Orf279 protein of amino acid sequence at least 95% identical to SEQ ID NO: 4.

In a specific embodiment, the present invention relates to an isolated nucleic acid wherein the sequence is depicted in SEQ ID NO: 1.

As demonstrated in the example, orf279 results from a recombination event in the genome. The 5′ part depicted in SEQ ID NO:2, corresponds to the 5′part of the gene encoding ATP synthase subunit 8 (the atp8 gene). On the contrary, the 3′ part of orf279, depicted in SEQ ID NO: 3, is specific to this gene, and it is called in the present application “orf279-unique region”.

The present invention also relates to a method for detecting orf279 DNA in wheat plant, seed or bulk of seeds, wherein the method comprises the step of extracting and isolating a DNA sample from a wheat plant and detecting orf279 DNA by specific means.

In a particular embodiment, the method for detecting orf279 DNA is performed in various lines or various varieties, more particularly in various wheat lines or varieties, for distinguishing the presence/absence of orf279 among different plants or varieties, and in particular among plants with CMS.

The presence of orf279 DNA means that the line or plant is harbouring a cytoplasm capable of inducing T-CMS. A line or plant harbouring such a cytoplasm may have a sterile phenotype or a fertile phenotype. In the latter case, such a fertile plant is likely to carry a functional restorer-of-fertility gene, “Rf”.

On the contrary, the absence of orf279 DNA means that the line or plant cannot exhibit T-CMS.

This particular embodiment is especially interesting for use in research for screening the diversity of wheat cytoplasms and in seed production for screening the quality of the sterile female genotype in a hybrid wheat system. The detection of orf279 DNA can be of interest for the creation and increase of parent lines and in seed production for the maintenance of the A-line and for the hybrid production.

In another embodiment, such method is of interest for identifying orf279 DNA in recombinant mitochondrial DNA. Recombinant mitochondrial DNA is obtained after the transfer of mitochondria from one plant to another plant, these plants belonging to the same specie or to different species. Such transfer can be achieved by any method that results in mixing mitochondria from two plant parents (i.e., protoplast fusion, grafting or sexual crosses in case bi-parental inheritance can be achieved). In the present invention the transfer occurs from a plant characterized by a T-CMS cytoplasm to another plant without a T-CMS cytoplasm.

The term “sterile female genotype” means that the plant which has this genotype is certain to harbour a male sterile cytoplasm.

The present invention also relates to a method for detecting orf279 RNA in a wheat plant, seed or bulk of seeds, wherein the method comprises the step of extracting and isolating an RNA sample from the wheat plant and detecting orf279 RNA by specific means.

In particular, one mean for the detection of orf279 DNA, RNA, are markers recognizing the recombination junction between atp8 and the “orf279-unique region”.

In another embodiment, means for detection are primers allowing the amplification of orf279 DNA, RNA. In particular, at least one of the primers hybridizes to the 3′ part of orf279 depicted in SEQ ID NO:3, sequence specific to orf279.

In particular, the forward primer and the reverse primer to amplify the orf279 DNA, RNA are chosen among:
forward primers: SEQ ID NO: 12, SEQ ID NO: 6, SEQ ID NO: 10; and
reverse primers: SEQ ID NO: 7, SEQ ID NO: 11, SEQ ID NO: 9; or are constituted by the forward primer SEQ ID NO: 8 and the reverse primer SEQ ID NO: 9.

In the present invention, the term DNA can include cDNA.

In a particular embodiment, the orf279 RNA expression level or pattern in a wheat line or plant can be quantified and compared to the orf279 RNA expression level and pattern of a reference plant material. The reference can be a sterile wheat line or a group of sterile wheat lines with a cytoplasm containing orf279 or a fertile line or a group of fertile plants. The fertile line can be either a maintainer line (considered as a negative control as it does not have a cytoplasm containing orf279) or a restored line with a cytoplasm containing orf279 and a nuclear genotype containing a restorer-of-fertility gene, Rf.

A similar level and pattern of orf279 RNA in the test plant compared to the sterile wheat line indicates that the plant is likely to present a CMS phenotype. A similar level and pattern of orf279 RNA in the test plant compared to a fertile wheat reference indicates that the plant is likely to present a fertile phenotype. As such, it is possible to screen or identify new Rf genes or assess the level of restoration of the fertility of the combination of different Rf genes in a genetic stack as described for example in WO2019086510.

Typically, but not limited to, the level of the orf279 RNA can be quantified with qRT-PCR or RNA-seq.

The present invention also relates to a method for detecting Orf279 protein in a wheat plant, seed or bulk of seeds, wherein the method comprises the step of extracting and isolating proteins and detecting Orf279 protein by specific means.

The means for detecting a protein in a protein extract are well-known by a person skilled in the art. In particular, Orf279 protein can be detected and quantified using immunological detection with an antibody directed towards an epitope located in the unique part of the Orf279 protein depicted in SEQ ID NO: 5. In particular, the detection of Orf279 protein is carried out by western blot, ELISA or immunostrip assay.

In a particular embodiment, the level or pattern of expression of Orf279 protein in a wheat line is quantified and compared to the level and pattern of Orf279 protein of a reference plant material. The reference can be a sterile wheat line or a group of sterile wheat lines with a cytoplasm containing orf279 or a fertile line or a group of fertile lines. The fertile line can be either a maintainer line (considered as a negative control as it does not have a cytoplasm containing orf279) or a restored line with a cytoplasm containing orf279 and a nuclear genotype containing a restorer-of-fertility gene, Rf.

A similar level and pattern of Orf279 protein in the test plant compared to the sterile wheat line indicates that the plant is likely to present a CMS phenotype. A similar level and pattern of Orf279 protein in the test plant compared to a fertile wheat reference indicates that the plant is likely to present a fertile phenotype.

Thus, the present invention also relates to a method for detecting orf279 DNA, orf279 RNA or Orf279 protein in a wheat plant, seed or bulk of seeds, wherein the method comprises the step of extracting a DNA or RNA or protein sample and detecting by means the orf279 DNA, orf279 RNA or Orf279 protein.

In a particular embodiment, said method comprises the steps of:
Detecting the sterile cytoplasm with a marker at the recombination junction between atp8 and SEQ ID NO:3, or within SEQ ID NO:3, or
Detecting the variation of orf279 expression in male sterile wheat plants.

The present invention also relates to means for Orf279 DNA, RNA or protein detection comprising:
a. molecular markers and primers recognizing orf279 recombination junction between atp8 and SEQ ID NO:3, such as the primers described above, or markers and primers recognizing SEQ ID NO:3; or
b. antibody recognizing Orf279, in particular an antibody directed towards an epitope located in SEQ ID NO:5.

The present invention also relates to a method for determining the sterility or fertility phenotype of a wheat plant, seed or bulk of seeds, comprising:
a step of extracting RNA and proteins
a step of detecting and quantifying orf279 RNA or Orf279 protein
a step of comparison of the orf279 RNA level (or pattern) or the Orf279 protein level (or pattern) with those quantified in sterile wheat lines containing orf279 and a fertile wheat line.

Means used for detecting and quantifying orf279 RNA and Orf279 protein are those previously described.

A similar level of orf279 RNA or Orf279 protein in a wheat plant compared to a sterile wheat line with a cytoplasm containing orf279 indicates that the plant will present a CMS phenotype.

This method allows the screening of diverse wheat cytoplasms or to screen the quality of sterile female genotype used in seed production in a hybrid wheat system.

The present invention also relates to a method for identifying a functional Rf gene encoding a protein able to bind to orf279 RNA, wherein said method comprises the steps of:
a. Predicting a target RNA sequence for protein encoded by each Rf gene identified in
a wheat plant genome according to the PPR code.
b. Aligning each predicted target RNA sequence with SEQ ID NO:3
c. Identifying the Rf gene encoding for a protein able to bind to a target RNA sequence that shows at least 95% identity to SEQ ID NO:3.
d. Optionally optimizing the sequence of the selected Rf protein by changing amino acids at position 5 and 35 of selected PPR motifs to improve match according to the PPR code with SEQ ID NO:3.

At step a, all the possible ribonucleotides are determined for each amino acid combination at position 5 and 35, according to the PPR RNA binding code. The target RNA sequence scoring highest for RNA binding to the corresponding PPR is selected. The target RNA sequence is 5 to 50 bases long and preferentially 10 to 20 bases long.

At step b, the predicted target RNA sequence is aligned to the orf279-unique region depicted in SEQ ID NO:3. Alignment can be done over the full length or a fragment of SEQ ID NO:3. Such fragment can be 5 to 50 bases long and preferentially 10 to 20 bases long.

At step c the target RNA sequence has at least 95, 96, 97, 98, 99 or 100% identity with SEQ ID NO: 3 over the aligned region.

In a particular embodiment, the method comprises a step e corresponding to the validation of the binding to orf279 RNA, wherein a plant with T-CMS cytoplasm is transformed with a vector expressing the Rf protein candidate, and fertility restoration is analyzed by phenotyping and/or by analyzing orf279 expression.

An alternative method for screening for a functional Rf gene encoding for a PPR protein able to bind to orf279 RNA, comprises the steps of:
a. Transforming a wheat plant with an expression cassette comprising a candidate Rf gene wherein said wheat plant has a sterile cytoplasm expressing the Orf279 protein.
b. Detecting the level and pattern of orf279 expression in the transformed plant.
c. Selecting the plant wherein the level or pattern of orf279 expression is altered compared to the non-transformed plant.
d. Identifying the functional Rf gene.

Transformation methods of a wheat plant are well-known by the person skilled in the art. For example, it can be carried out using an Agrobacterium tumefaciens-mediated approach or a biolistic approach.

In this method, Rf candidate genes, are screened for their capacity to alter the orf279 RNA or Orf279 protein level or pattern compared to a wheat line with a sterile cytoplasm expressing orf279. An alteration can be a decrease of the RNA or protein level in one or more tissues of the plant.

Rf candidate genes able to restore fertility are selected.

Another aspect of the present invention relates to a method for the design and optimization of a synthetic PPR protein capable of binding orf279 RNA, and preventing its expression, whereby said synthetic PPR protein restores fertility.

According to the present invention, the expression “preventing expression of orf279 RNA” means induction of RNA cleavage, RNA degradation, or inhibition of translation.

In a particular embodiment, this method comprises the steps:
a. Identifying a target RNA sequence of interest in SEQ ID NO:3, said target RNA sequence is 5 to 50 bases long and preferentially 10 to 20 bases long.
b. assigning to each target RNA base selected from the group comprising adenine (A), guanine (G), cytosine (C), and uracil (U), a pair of amino acids according to the PPR RNA binding code
c. designing a synthetic PPR sequence comprising PPR RNA binding motifs (each containing an amino acid pair defined in step b) capable of binding the target RNA sequence.

In another particular embodiment, this method comprises the optimization of a candidate Rf protein able to restore the cytoplasmic fertility, more particularly identified as previously described or as described in example C. This optimization aims to improve binding of the Rf protein to the orf279 RNA.

In a particular embodiment, the optimization is carried out as described in FIG. 5. The amino acids at positions 5 and 35 of each motif of the PPR are listed, indicating the number of the motif, and indicating below the RNA sequence. Amino acids at position 5 and/or 35 that do not make a perfect match with the RNA binding site according to the PPR code are changed according to this code in order to improve the binding to the RNA sequence.

The PPR code is described in the international application WO2013/155555A1.

The present invention also concerns synthetic PPR proteins capable of binding orf279 RNA obtained from the method above.

In a particular embodiment, a PPR protein capable of binding orf279 RNA is depicted in SEQ ID NO: 22.

In particular, a synthetic PPR protein capable of binding orf279 RNA, preventing expression of orf279 and thus restoring fertility.

The restoration of fertility can be easily checked by introducing a vector expressing the synthetic PPR into a wheat plant with T-CMS cytoplasm, and then proceeding to fertility restoration phenotyping assays as described in the example, part B.

In another aspect, the present invention relates to a method for obtaining a fertile wheat plant by transforming said plant with a vector expressing a synthetic PPR binding orf279 and preventing its expression. The vector comprises a recombinant expression cassette comprising a nucleic acid sequence encoding the synthetic PPR, downstream of a promoter functional in plants.

In a specific embodiment, such method also comprises transforming said plant with a vector expressing a PPR binding orf256.

The present invention also concerns a plant expressing a synthetic PPR binding orf279 obtained as previously described. Such a plant is therefore of fertile phenotype.

The term “promoter” as used herein refers to a region of DNA upstream of the coding sequence (upstream of start codon) and including DNA regions for recognition and binding of RNA polymerase and other proteins to initiate transcription before the start codon. Examples of constitutive promoters useful for expression include the 35S promoter or the 19S promoter (Kay et al, 1987), the rice actin promoter (McElroy et al, 1990), the pCRV promoter (Depigny-This et al, 1992), the CsVMV promoter (Verdaguer et al. 1996), the ubiquitin 1 promoter of maize (Christensen and Quail, 1996), the regulatory sequences of the T-DNA of Agrobacterium tumefaciens, including those from the genes encoding mannopine synthase, nopaline synthase, octopine synthase.

Promoters may be “tissue-preferred”, i.e. initiating transcription in certain tissues or “tissue-specific”, i.e. initiating transcription only in certain tissues. Examples of such promoters are DHN12, LTR1, LTP1 specific for the embryo, SS1 specific for the phloem, OSG6B specific for the tapetum (Gotz et al 2011 and Jones 2015).

Other suitable promoters could be used. It could be an inducible promoter, or a developmentally regulated promoter. An “inducible” promoter initiates transcription under some environmental control or can be stress-induced, like for example the abiotic stress-induced RD29, COR14b (Gotz et al, 2011).

Typically, the promoter is functional in the nucleus.

Constitutive promoters may be used, such as the ZmUbi promoter, typically the ZmUbi promoter of SEQ ID NO:16, or the promoter CaMV35S. Finally, the promoters of SEQ ID NO: 26, SEQ ID NO:27 and SEQ ID NO:28 corresponding to pTaRFL46, 79 and 104 can also be used.

In particular, constitutive promoters used in the present application are: proZmUBI_intUBI depicted in SEQ ID NO:14, proOsActin_intOsActin, pro VirCsVMV, pro Vir35S, pro35S_intZmUBI.

Another aspect of the invention concerns a recombinant expression cassette comprising the nucleic acid sequence encoding for Orf279 of SEQ ID NO:1, downstream of a promoter functional in plants and a sequence encoding a mitochondrial transit peptide.

The mitochondrial transit peptide allows to address the peptide to the mitochondria. Huang et al. (2009) describes the main characteristics of plant mitochondrial transit peptides.

In particular, the mitochondrial transit peptide is a sequence from Oryza sativa, and more particularly it corresponds to OsPPR_02g02020 depicted in SEQ ID NO: 19, or Os01g49190 depicted in SEQ ID NO: 20.

Such a recombinant cassette can be used for transforming a plant. Thus, the present invention also relates to a plant expressing a recombinant expression cassette comprising the nucleic acid sequence encoding for Orf279 downstream of a promoter functional in plants and a mitochondrial transit peptide.

In a particular embodiment, said plant is wheat.

The present invention also relates to a method for obtaining a sterile plant by transforming said plant with a recombinant expression cassette comprising the nucleic acid sequence encoding for Orf279 downstream of a promoter functional in plants and a mitochondrial transit peptide.

In a particular embodiment, said plant is also transformed with recombinant expression cassette comprising the nucleic acid sequence encoding for Orf256 downstream of a promoter functional in plants and a mitochondrial transit peptide. In a more particular embodiment, said plant is wheat.

In order to obtain a fertile plant, in particular a wheat plant, from a plant having a cytoplasmic male sterility and having the gene orf279, it is possible to use known means allowing to decrease the transcription of the gene and/or the translation of the orf279 RNA.

In another aspect, the present invention concerns a method for obtaining a fertile wheat plant by transforming said plant with a recombinant expression cassette comprising a gene (or genes) encoding an orf279 DNA/RNA binding or editing complex, said orf279DNA/RNA binding or editing gene is cloned downstream of a promoter functional in plants and a mitochondrial transit peptide.

The orf279 DNA/RNA binding or editing complex is a DNA or RNA editing tool which is able to either (1) disrupt the orf279 gene directly in the mitochondrial genome or (2) to reduce expression of the orf279 transcript.

Known genome editing tools can be used to target the corresponding orf279 gene within the wheat plant nuclear and mitochondrial genomes by deletion, insertion or partial or total allele replacement at the corresponding locus. Such genome editing tools include without limitation targeted sequence modification provided by double-strand break technologies such as, but not limited to, meganucleases, zing finger nuclease, TALENs (WO2011072246) or CRISPR CAS system, including CRISPR Cas9 (WO2013181440) and CRISPR Cas13a, Cpf1 or their next generations based on double-strand break technologies using engineered nucleases. An RNA editing factor could be designed and used to introduce premature translation termination by introduction of a STOP codon in the coding sequence of orf279 transcripts in wheat mitochondria.

In another aspect, the present invention concerns a method for detecting sterile plants harbouring a orf279 T-CMS cytoplasm or fertile plants harbouring a normal cytoplasm wherein the method comprises the steps of:
a) extracting a DNA or RNA sample from the plants
b) detecting the presence or absence of orf279 T-CMS sequence by PCR amplification with suitable pair of primers, and optionally, detecting the presence or absence of normal cytoplasm sequence by PCR amplification with suitable pair of primers.
c) determining the fertile or sterile status of the plants.

Suitable pair of primer for amplifying orf279 T-CMS can be for example the pair of primers SEQ ID NO: 52 and 54, or variant thereof. Other suitable pair of primers can be obtained by using a forward primer selected from the sequence SEQ ID NO: 12, SEQ ID NO: 6, SEQ ID NO: 10 or SEQ ID NO: 8, and a reverse primer selected from the sequence SEQ ID NO: 7, SEQ ID NO: 11 or SEQ ID NO: 9, preferably by using the pair of primers obtained with forward primer SEQ ID NO: 8 and reverse primer SEQ ID NO: 9.

Suitable pair of primer for amplifying normal cytoplasm sequence can be for example the pair of primers SEQ ID NO: 53 and 54, or variant thereof.

Step c) of determining the fertile or sterile plant status can be performed by detecting the presence or absence of a PCR amplification signal. Particularly, sterile status depends on the presence of an amplification signal using specific primers capable of amplifying of orf279 T-CMS sequence, while fertile status is determined by the absence of said amplification signal.

Optionally, a step of amplifying normal cytoplasm sequence with suitable pair of primers can be performed as positive control to confirm the fertile plant status.

Of course, the skilled person may use variant primers as identified above, said variant primers or nucleic acid probes having at least 90%, and preferably 95% sequence identity with any one of the primers as identified above.

Percentage of sequence identity as used herein is determined by calculating the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. For example, nucleic acid sequences may be aligned using the BLAST 2 sequences (B12seq) using BLASTN algorithms (www.ncbi.nlm.nih.gov).

In a further aspect, the present invention concerns a diagnostic marker for determining the presence or absence of orf279 comprising the pair of primers SEQ ID NO: 52 and 54 to amplify orf279 T-CMS sequence and, optionally, the pair of primers SEQ ID NO: 53 and 54to amplify the normal cytoplasm sequence.

The orf279 diagnostic marker to follow the presence of the T. timophevii cytoplasm during the creation or conversion and increase of parent lines and the hybrid production can be used for the following activities:
Breeding schemes for parental lines creation:
   Conversions by back-cross of the T. timophevii cytoplasm in A lines and R lines, if the restorer is alloplasmic, meaning if the R line carries the T-CMS cytoplasm. Control of Orf279 presence in single seeds, bulk of seeds or plants
   Creation of R lines by DH, SSD, pedigree breeding or any other breeding scheme, if the restorer is alloplasmic, meaning if the R line carries the T-CMS cytoplasm. Control of Orf279 presence in single seeds, bulk of seeds or plants
Production for research of commercial purposes
   Control of single seeds, bulks of seeds or plants of the A line, for maintenance, hybrid production or DUS assessment
   Control of single seeds, bulks of seeds or plants of the R line if the restorer is alloplasmic, meaning if the R line carries the T-CMS cytoplasm
   Control of single seeds, bulk of seeds or plants of T-CMS hybrids. The marker allows to measure the contamination by non alloplasmic grains (any wheat line on wheat cytoplasm) within the hybrid lots. This can be used to control the purity and hybridity levels of hybrid seed lots.
   Control of F1 seed lots sent for DUS trials and official trials. The marker allows to verify if the hybrid is alloplasmic, an essential step to ensure fertility of the hybrid.

### EXAMPLES

### A—Production and Phenotyping of Rf1 and Rf3 Transformants

Fine mapping of the genomic regions harbouring Rf1 and Rf3 restorer genes in wheat was performed and Rfl genes present in the Rf1 and Rf3 interval in the IWGSC RefSeqv1.0 reference genome were identified. In parallel, Rfl genes present in Triticum timopheevii and wheat Rf1, Rf3 or maintainer accessions were enriched and sequenced by targeted Rfl capture. Both mapping and capture analysis allowed to predict candidate Rf1 and Rf3 restorer genes. Restoring capabilities of Rf1 and Rf3 candidate genes were assessed by transgenic approaches.

The nucleic acid encoding TaRFL79 protein of amino acid sequence depicted in SEQ ID NO: 15, was identified as the Rf1 restorer gene. The nucleic acid encoding TaRFL29a protein of amino acid sequence depicted in SEQ ID NO: 25 was identified as the Rf3 restorer gene. Each were separately adapted and cloned via a Golden Gate reaction into the destination binary plasmid pBIOS10746.

The following expression elements were used for each construct: the constitutive Zea mays ubiquitin promoter (proZmUbi depicted in SEQ ID NO: 16) associated with the Zea mays ubiquitin intron (in (ZmUbi, depicted in SEQ ID NO: 17 Christensen et al 1992) and a 3′ termination sequence of the gene encoding a sorghum heat shock protein, terSbHSP (accession number: Sb03g006880), depicted in SEQ ID NO: 18.

The recombinant constructs of TaRFL79 and TaRFL29a are respectively depicted in SEQ ID NO: 29 and SEQ ID NO: 30.

All the binary plasmids described above were transformed into Agrobacterium EHA105 strain. BGA CMS*Fielder wheat as well as conventional Fielder cultivar were transformed with those Agrobacterium strains as described by WO2000/063398. Wheat transgenic events were generated for each of the constructs.

BGA CMS*Fielder is a Fielder maintainer line carrying T-CMS cytoplasm. This line was constructed in order to combine both sterility high transformation efficiency and tissue regeneration. The BGA CMS*Fielder plants are sterile.

Fertility restoration phenotyping assays were performed on the different events as following. All wheat transgenic plants generated above and control fertile plants were grown in a glasshouse under standard wheat growth conditions (16 h of light period at 20° C. and 8 h of dark period at 15° C. with constant 60% humidity) until control grains of the wild type Fielder cultivar reached maturity stage.

Fertility of the transgenic plants was evaluated by counting the number of seeds and empty glumes per spike on each plant and comparing with the wild type Fielder and BGA CMS*Fielder control plants. Plants were also evaluated by observing anther extrusion.

16 transformed CMS-Fielder plants overexpressing the TaRFL79 sequence under the ZmUbi promoter derived from 11 independent transformation events and 36 transformed CMS-Fielder plants overexpressing the TaRFL29a sequence under the ZmUbi promoter derived from 19 independent transformation events were analyzed.

100% to 92% respectively of the analyzed plants present restoration of male fertility while 100% of untransformed CMS-Fielder plants grown in parallel are fully sterile with no anther extrusion and no seed produced, and 100% of WT-Fielder plants are fertile.

### B—Molecular Characterization of the Rf1 and Rf3 Transformants

### 1—Material and Method

### RNA Analyses

RNA was extracted from the plants with the RNeasy Plant Mini kit (Qiagen) according to manufacturer's instructions. For transgene expression analyses cDNA was synthesized with SuperScript™ III Reverse Transcriptase (Invitrogen) kit and the amplification was performed with primers P1, P2 and P3 listed in table 1.

TABLE 1
sequence of the different primers used in the
present analysis

| | | SEQ |
|---|---|---|
| | | ID |
| Primer Name | Sequence 5′->3′ | NO |
| GSP1 | GGA TTT GCC CGC AAA TGG TTG ATC | 31 |
| GSP2 | GAT TAC GCC AAG CTT AAG AAT CAG | 32 |
| | AAT TAC TGA GCT ACC CCG CTC TT | |
| P1 | ATGACAAATATGGTTCGATGGC | 33 |
| P2 | GCTTGGGGATCCTGAATC | 34 |
| P3 | GCTGTCACTAGAACGGACC | 35 |
| Ta_Actin_F | GCCACACTGTTCCAATCTATGA | 36 |
| Ta_Actin_R | TGATGGAATTGTATGTCGCTTC | 37 |
| WORF256_211_ | ATCCCCAAGCTCTAGCTCATTTAG | 38 |
| 806_for | | |
| WORF256_211_ | GGGGGCTGGAAGAGAAAAGAAT | 39 |
| 806_rev | | |

Northern Blot 5-10 μg of total RNA was separated on 1.2% denaturing agarose gel and transferred onto Hybond N+membrane (Amersham). Northern blotting was performed overnight in 10×SSC (1.5 M sodium chloride and 150 mM trisodium citrate pH 7.0) buffer. The membrane was pre-hybridised in PerfectHyb™ Plus Hybridization Buffer (Sigma) for 2 hrs at 65° C. The biotin-labelled probes were hybridized overnight in hybridization buffer. After 3 washing steps with wash solution containing decreasing concentration of the SSC buffer supplemented with SDS the probe signal was detected using the Chemiluminescent Nucleic Acid Detection Module Kit (ThermoFisher) and ImageQuant™ imager (GE Healthcare). For probe synthesis of Actin and orf256 RNA, the DNA fragment was amplified with primers given in table 1. The reaction products were cloned into the pGEM®-T Easy (Promega) vector and confirmed by PCR and sequencing at Macrogen (Macrogen, South Korea). The RNA probe was synthesized with the MAXIscript™ SP6/T7 Transcription Kit (Ambion) and the pGEM®-T Easy plasmid as a template and biotin labeled analog of cytidine triphosphate (CTP) (Roche).

### Rapid Amplification of CDNA Ends (5′-RACE)

1 μg of total RNA was used for cDNA synthesis and amplification of 5′ ends using the SMARTER@RACE 5′3′ Kit following manufacturer's instructions (Takara). PCR products were gel purified, cloned into pGEM®T Easy (Promega) and sequenced at Macrogen (Macrogen). Gene specific primer sequences (GSP1 for orf279 and GPS2 for orf256) are given in table 1.

### Mitochondrial DNA Sequencing and Assembly

The extraction of mitochondrial DNA was preceded by enrichment of mitochondrial fractions from seven-day-old wheat coleoptiles grown on vermiculite in a growth cabinet at 22° C. in darkness. For mitochondrial isolation and DNA extraction previously described protocols were adapted (Huang et al., 2004, Triboush et al., 1998). The obtained DNA (50 ng) was ultrasonicated to 550 bp fragments with a Covaris S220 focused ultrasonicator (Covaris, USA). The libraries were prepared with the TruSeq® Nano DNA LT Sample Preparation Kit—Set A (Illumina, USA). The normalized and pooled libraries were used for sequencing on a MiSeq desktop sequencer (Illumina, USA) with the MiSeq® Reagent Kit v3 (600 cycles) (Illumina, USA). Overlapping paired-end reads were merged using the software FLASH [version 1.2.7] and merged reads were assembled using Velvet [version 1.2.08], with a k-mer value of 91 and a coverage cut-off of 20. To identify ORFs unique to the T. timopheevii mitochondrial genome, reads from the T-CMS line were mapped to the T. aestivum mitochondrial genome reference (DNA Database accession no. AP008982, Ogihara et al., 2005) to filter out the reads that are common to both T. timopheevii and T. aestivum genomes. The remaining unmapped reads were re-assembled into contigs with Geneious software (www.geneious.com/).

### RNAseq Analysis of BGA CMS*Fielder and Rf1 and Rf3 Transformants

RNA was extracted from BGA CMS*Fielder and Rf1 transformants using the RNAeasy Plant Mini Kit (Qiagen) and its quality was estimated on an Agilent 4200 tape station (Agilent). 3 μg of total RNA was sent to Macrogen for NGS sequencing. The libraries were performed with the TruSeq Stranded Total RNA Ribo Zero Samples Prep Kit (Illumina) and sequenced on a Hiseq4000 platform (Illumina) with 100 bp paired-end sequencing kit (Illumina). Reads were adapter-trimmed and mapped to the T. timopheevii mitochondrial genome (NC_022714) with BBMap (Bushnell B. sourceforge.net/projects/bbmap/). Multipmapped reads were distributed randomly between the best-matching sites and rRNA regions were masked (because rRNA depletion was inconsistent across samples). Regions identical to plastid DNA were masked to avoid cross-mapped plastid reads and read depth was normalized by dividing by mean coverage depth excluding the masked regions.

### 2—Results

### A—Processing of Orf256 Does Not Correlate With Fertility Restoration in T-CMS Wheat

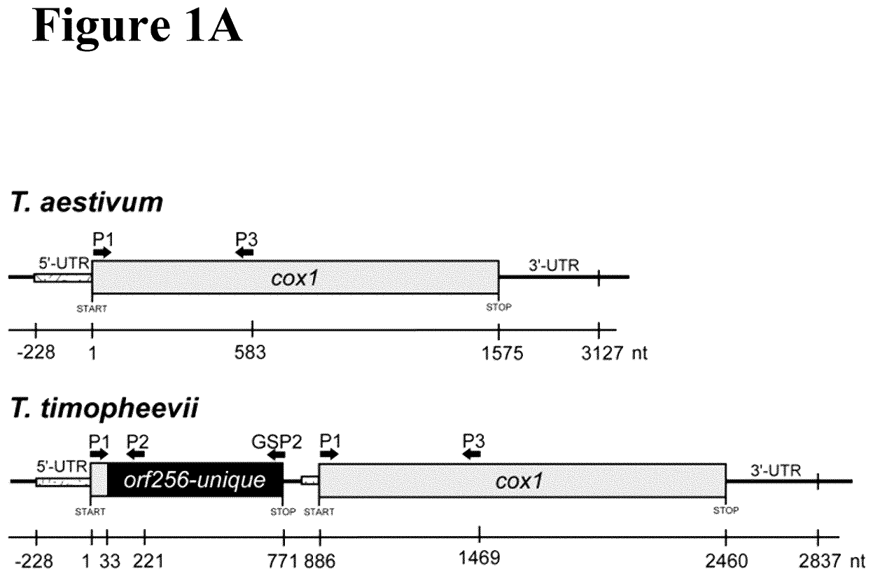

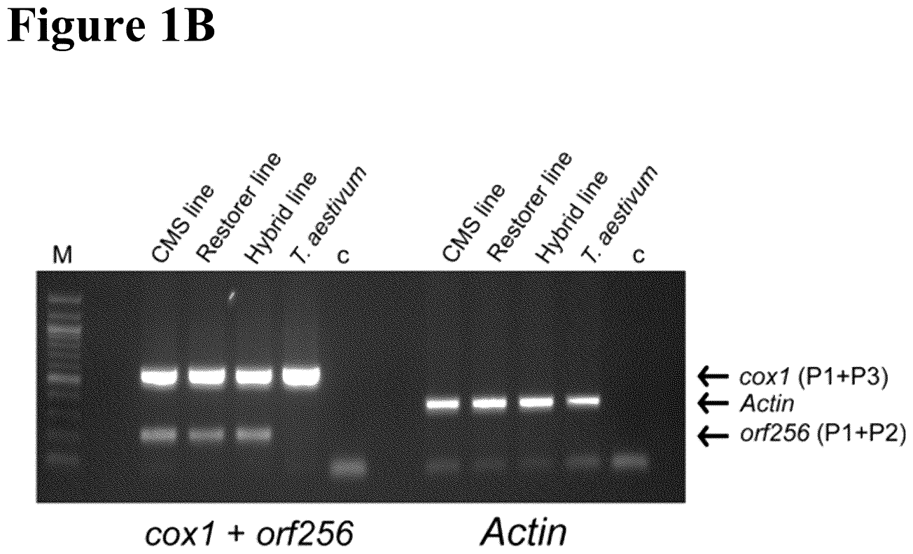

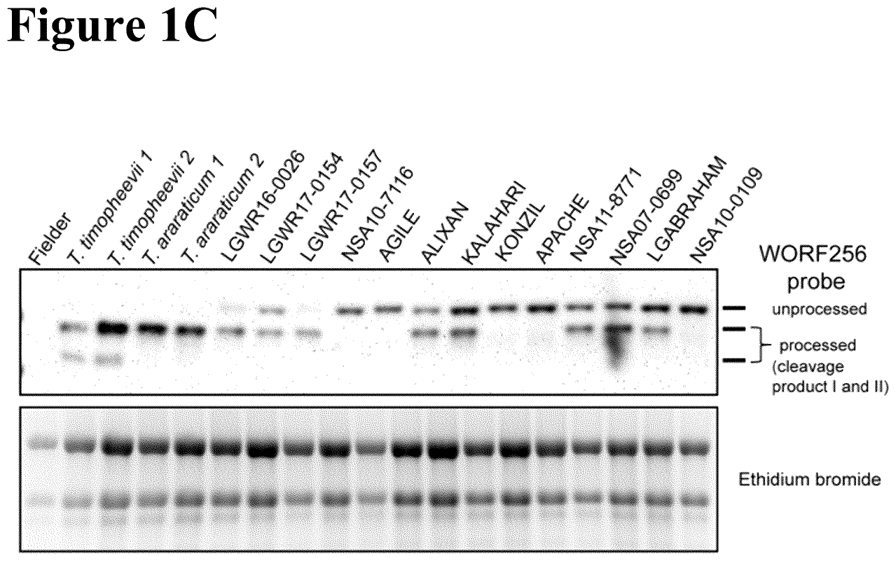

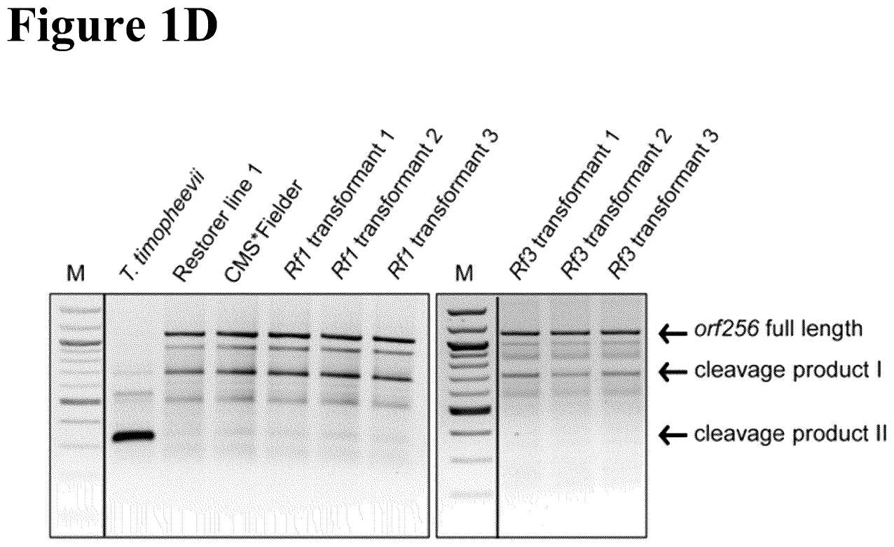

A previous study indicated that fertility restoration of T-CMS plants is correlated with the expression of Orf256 protein (Song and Hedgcoth, 1994A) and that the nuclear background influenced the level of the orf256 transcript in wheat accessions (Song et al., 1994B). To analyse the expression and processing pattern of orf256 in wheat genotypes carrying either T. aestivum or T. timopheevii cytoplasm and with different restoring capabilities, total RNA was extracted and RT-PCR as well as northern blot analysis with an orf256-specific probe was performed (FIGS. 1A-1D). The RT-PCR results show that orf256 RNA is quite abundant across plant accessions and levels are independent of the presence of a restorer gene (FIG. 1A). The northern blot results revealed that the processing of orf256 did not correlate with the restoration of fertility phenotype as even in wheat lines known not to carry a restorer gene, a processing of orf256 RNA was observed (FIG. 1C). Indeed, several lines including Alixan, Kalahari, Lgabraham known to not carry a restorer gene show processing of orf256 at cleavage site I as compared to lines T. timopheevii or LGWR16-0026, LGWR17-0154 and LGWR17-0157 known for carrying restorer genes.

To analyze the processing of orf256 in the different genotypes as well as in the Rf1 and Rf3 transformants in more detail, Rapid Amplification of cDNA Ends (5′-RACE) was performed (FIG. 1D). Cleavage of orf256 is observed in T. timopheevii and in T-CMS plants with a maintainer genotype BGA CMS*Fielder plants (FIG. 1D). In addition, a second cleavage site in orf256 was found only in T. timopheevii in agreement with the northern blot result (FIG. 1C).

### B-Identification of Orf279 as the Genetic Basis of CMS in T-CMS Wheat

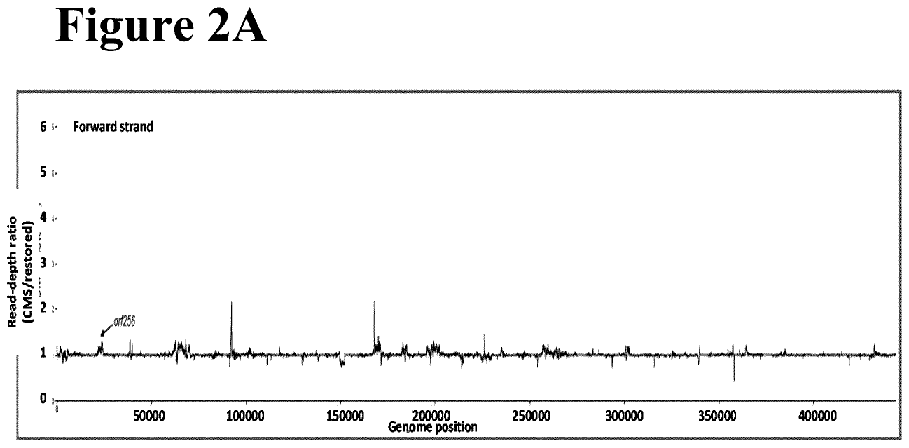

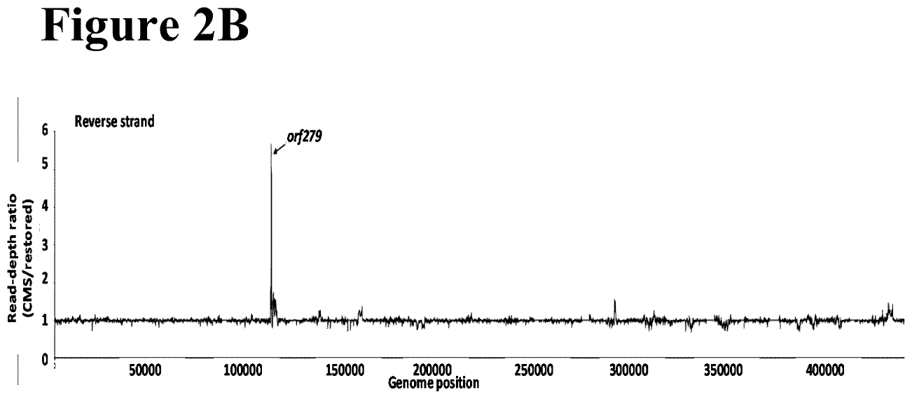

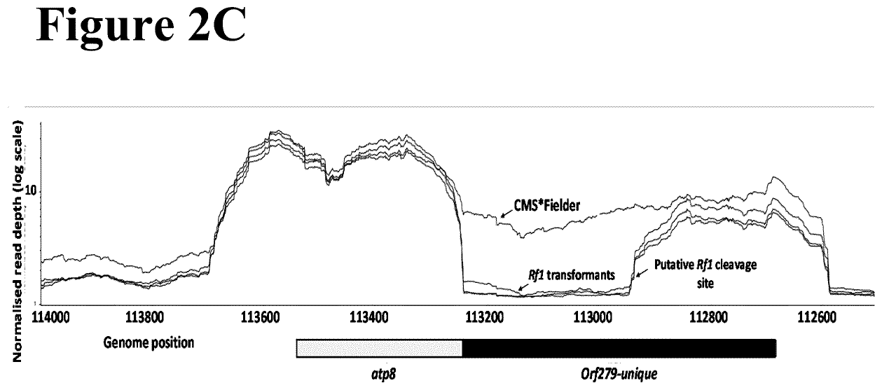

As the processing/cleavage of orf256 in T-CMS mitochondria does not correlate with the presence of either Rf1 or Rf3 restorers, the T. timopheevii mitochondrial DNA was extracted and sequenced to look for the presence of other chimeric orfs that could be the molecular basis for CMS in T. timopheevii. Mitochondrial DNA was sequenced on Illumina MiSeq platform and 17 contigs present in T. timopheevii and absent in the T. aestivum mitochondrial genome were identified (Table 2). 25 candidate ORFs corresponding to the best ORFs identified between two STOP codons and encoding peptides longer than 100 amino acids were identified (Table 2). Orf256 was found to be encoded within contig 11 as ORF5 (Table 2). The remaining uncharacterised 24 ORFs were screened for regions homologous to other genes from the T. timopheevii mitochondrial genome or other sequenced plant genomes by using blastn (https://blast.ncbi.nlm.nih.gov/) (Table 2). The searches revealed that two ORFs were already identified to be encoded by the mitochondrial genomes of Oryza sativa (contig 1, orf27=orf194) and Zea mays (contig 5, orf21=orf296), respectively. Subsequent RNAseq analysis of RNA samples from BGA CMS*Fielder as well as Rf1 and Rf3 transformants revealed that the biggest proportional reduction in expression was observed within the 1.1 kb region of contig_4_orf13 (FIG. 2A and B). This region was found to encode a protein composed of 279 amino acids and thus was named orf279. In the Rf1 and Rf3 transformants, the orf279 transcript is cleaved; the 5′ end is degraded (preventing translation) but the 3′ end persists (FIG. 2C). Most of the reads mapping to the 5′ region of the ORF and upstream are probably from the other (complete) copy of atp8 present in the mitochondrial genome (FIG. 2C).

TABLE 2
List of contigs and ORFs found as present in the T. timopheevii
mitochondrial genome and absent in T. aestivum genome.

| | | Location in the | | | | | |
|---|---|---|---|---|---|---|---|
| | | mitochondrial | | | | no. of | |
| Contig | | genome | | in close | no. of reads | identified ORFs | |
| No. | length | from | too | proximity to | assembled | within the contig | best orf |
| 1 | 6 036 | 438 186 | 443 419 | 1476 bp upstream | 3 626 | 52 | ORF 27 (frame 1) |
| | | | | of CobA | | | |
| 2 | 7 053 | 205 909 | 212977 | 1173 bp upstream | 30 739 | 70 | ORF 21 (frame 2) |
| | | | | of atp8 | | | |
| 3 | 4 901 | 23 198 | 28114 | downstream of | 14 927 | 45 | ORF 1 (frame 2) |
| | | | | cox1 | | | ORF 7 (frame 1) |
| | | | | | | | ORF 16 (frame 1) |
| 4 | 3 044 | 110 864 | 113 743 | encompasses p- | 11 839 | 23 | ORF 13 (frame 3) |
| | | | | gene atp8 | | | |
| 5 | 3 495 | 295 117 | 315 783 | 1760 bp | 10 524 | 31 | ORF 19 (frame 2) |
| | | | | upstream of trnF | | | ORF 21 (frame 1) |
| 6 | 1 920 | 248 389 | 250 306 | between orf-240 | 9 389 | 17 | ORF 10 (frame 3) |
| | | | | and p-ccmC | | | |
| 7 | 3 239 | 155 448 | 158 643 | encompasses C- | 8 448 | 29 | ORF 22 (frame 1) |
| | | | | terminus of CobB | | | ORF 24 (frame 3) |
| | | | | | | | ORF 19 (frame 3) |
| 8 | 2 145 | 171 240 | 174 141 | between 236 bp- | 8 344 | 23 | ORF 2 (frame 1) |
| | | | | p-rpl16 and 573 | | | |
| | | | | bp-p-orf256 | | | |
| 9 | 2 297 | 430 951 | 433 248 | 1478 downstream | 7 406 | 16 | ORF 10 (frame 3) |
| | | | | of cobA | | | |
| 10 | 2 378 | 152 704 | 154 048 | 260 bp downstream | 6 255 | 21 | ORF 11 (frame 2) |
| | | | | of atp9 | | | |
| 11 | 1 982 | 22 510 | 23 949 | upstream of cox1 | 5 545 | 9 | ORF 5 (frame 3) |
| | | | | | | | ORF 9 (frame 3) |
| 12 | 1 810 | 15 652 | 16 704 | 3,106 bp | 4 959 | 17 | ORF 1 (frame 1) |
| | | | | upstream of | | | |
| | | | | orf256 | | | |
| 13 | 1 531 | 15 652 | 16 704 | downstream of | 4 185 | 16 | ORF 14 (frame 3) |
| | | | | rps7 | | | |
| 14 | 1 911 | 132 568 | 134 477 | 2306 bp dpwnstream | 4 120 | 18 | ORF 10 (frame 1) |
| | | | | of rpl16 | | | |
| 15 | 1 748 | 165 189 | 166 960 | 1123 bp upstream | 3 940 | 20 | ORF 15 (frame 2) |
| | | | | of rps3 exon 1 | | | ORF 8 (frame 2) |
| 16 | 1 409 | 221 333 | 222 752 | 2022 upstream of | 3 836 | 14 | ORF 11 (frame 3) |
| | | | | rpl5 | | | ORF 5 (frame 3) |
| 17 | 1 320 | 172 302 | 172 622 | encompasses p- | 1 773 | 12 | ORF 2 (frame 2) |
| | | | | rpl16 (R8) and | | | |
| | | | | rps2 (R7) | | | |

| Contig | | | | | Repeat | cp genome |
|---|---|---|---|---|---|---|
| No. | length | orientation | best hit | NCBI | region | hit |
| 1 | 411 | forward | hypothetical protein | ref\|XP_005502205.1 | R1 | no |
| | | | [Oryza sativa Indica | | | |
| | | | Group] | | | |
| 2 | 507 | forward | no significant similarity | | no | no |
| | | | found | | | |
| 3 | 690 | forward | cox1 C-terminus | | | |
| | 351 | forward | no significant similarity | | no | no |
| | | | found | | | |
| | 321 | forward | no significant similarity | | | |
| | | | found | | | |
| 4 | 924 | reverse | p-gene atp8 | | R9 | no |
| | | | (T. timopheevi) or atp8-l | | | |
| | | | [Triticum aestivum] | | | |
| | | | YP_398423 partially | | | |
| 5 | 699 | reverse | hypothetical protein | | no | no |
| | | | YYE_00847 [Plasmodium | | | |
| | | | vinckei vinckei] | | | |
| | 420 | reverse | hypothetical protein | | | |
| | | | (mitochondrion) [Zea | | | |
| | | | mays subsp. mays] | | | |
| 6 | >268 | forward | no significant similarity | | R2, R4 | no |
| | | | found | | | |
| 7 | 375 | reverse | puroindoline B [Triticum | | R1 | no |
| | | | timopheevii subsp. | | | |
| | | | timopheevii] see | | | |
| | | | alignment | | | |
| | 366 | reverse | no significant similarity | | | |
| | | | found | | | |
| | 249 | reverse | CobB C-terminus | | | |
| 8 | 354 | forward | similarity to rps2 (maybe | gb\|AGI48804.1 | R7 | no |
| | | | pseudo rps2??) see | | | |
| | | | contig 13 | | | |
| 9 | 249 | reverse | slight similarity to | gb\|KDQ56778.1\| | no | no |
| | | | hypothetical protein | | | |
| | | | JAAARDRAFT_207845 | | | |
| | | | [Jaapia argillacea MUCL | | | |
| | | | 33604] Sequence ID: | | | |
| 10 | 1 773 | reverse | photosystem I P700 | NP_114259.1 | R6 | yes, |
| | | | chlorophyll a apoprotein | GI:14017572 | | insertion in |
| | | | A1 [Triticum aestivum] | | | T. timopheevi |
| | | | | | | genome |
| 11 | 771 | forward | ORF256 | | | no |
| | >543 | forward | cox1 N-terminus | | | |
| 12 | 273 | forward | no significant similarity | | no | no |
| | | | found | | | |
| 13 | >458 | reverse | similarity to rps2 see | | R7 | no |
| | | | contig 8 | | | |
| 14 | 381 | forward | no significant similarity | | no | no |
| | | | found | | | |
| 15 | 348 | reverse | no significant similarity | | R1 | no |
| | | | found | | | |
| | 204 | forward | no significant similarity | | R1 | |
| | | | found | | | |
| 16 | 249 | reverse | no significant similarity | | no | no |
| | | | found | | | |
| | 216 | forward | no significant similarity | | | |
| | | | found | | | |
| 17 | 594 | forward | rpl-16 (partially) and | | R8 and | no |
| | | | rps2 see contig 8 and 13 | | R7 | |

### C-Processing of Orf279 Correlates With the Fertility Restoration Phenotype of Rf1 and Rf3 Transformants

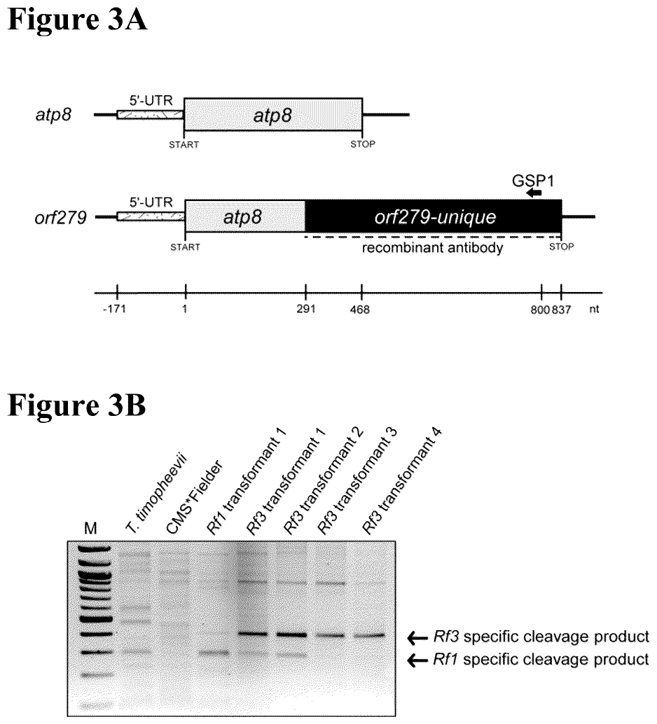

Detailed analysis of Orf279 revealed that the first 96 amino acids are identical with the N-terminus of the ATP synthase subunit 8 (FIG. 3A). In addition, the 171 nt upstream of the translation start are identical with the 5′ UTR region of the atp8 gene (FIG. 3A).

To analyze the processing of the orf279 transcripts a 5′-RACE analysis was performed. A major amplification product of ˜300 nt was detected with GSPI primers in the Rf1 transformants, whereas in Rf3 transformants a major amplicon of ˜400 nt was found (FIG. 3B). In agreement with the origin of the Rf1 restorer from T. timopheevii and the Rf3 restorer gene from T. aestivum, only the Rf1-specific amplicon was detected in T. timopheevii and not the Rf3-specific amplicon. Neither of these two amplicons was detected in the BGA CMS*Fielder sample (FIG. 3B). These results indicate that: (1) orf279 is processed at two different sites-cleavage induced by Rf3 generally occurs upstream of the RNA cleavage induced by Rf1; (2) the endonuclease attracted by Rf3 may sometimes skip the first cleavage site and continue to cleavage site targeted by Rf1.

### C—RF Protein Optimization for Improving Suppression of Orf279 Expression

### 1—Designing and Obtaining Synthetic Rf Proteins SR Orf279 and SR Orf256

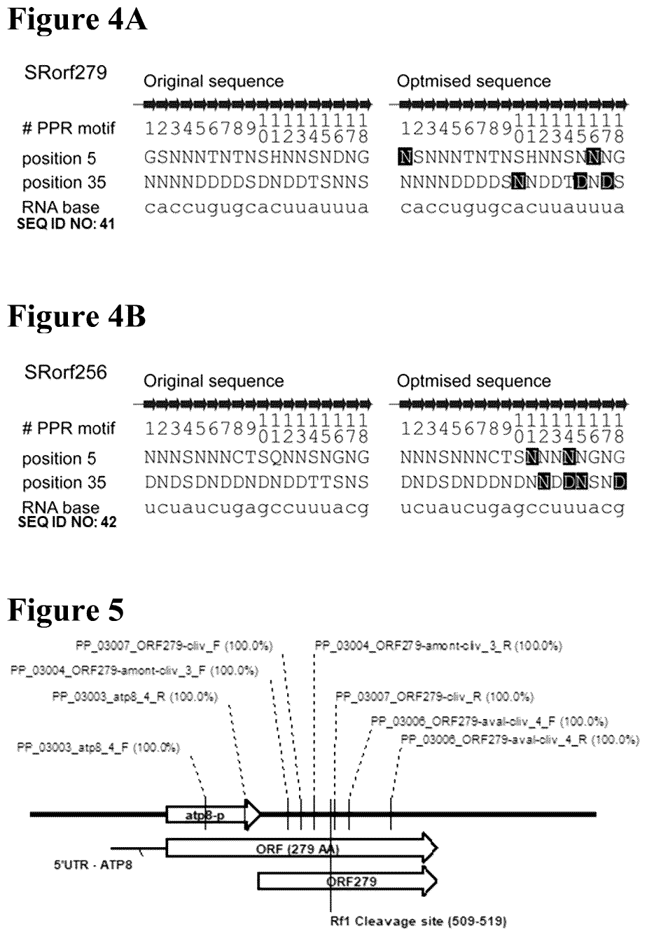

A library of 2973 RFL proteins identified by targeted sequence capture from 52 wheat accessions as well as RFL proteins annotated in the Refseqv1.1 Chinese Spring wheat genome (IWGSC) was screened for RFL sequences which according to the PPR code described in Barkan et al. (2012) and patent application WO2013155555 scored highest for RNA binding within orf279 or orf256. The best candidates were analyzed for the presence of a mitochondrial targeting sequence with Predotar (Small et al., 2004) and TargetP (Emanuelsson et al., 2007). The best candidates for either Synthetic Restorer binding to orf279 (SRorf279) or Synthetic Restorer binding to orf256 (SRorf256) were optimized by altering the amino acid combinations at position 5 and 35 according to the PPR code in the PPR motifs that did not form a perfect match with the RNA binding site (FIG. 4). Optimized SRorf279 is depicted in SEQ ID NO: 22 and optimized SRorf256 is depicted in SEQ ID NO: 21. The expression of these optimized sequences can be fused with the expression of a tag sequence depicted in SEQ ID NO: 40, in C-terminal of the optimized sequences.

### 2—Cloning and Transformation of Optimized SR Orf279 and SR Orf256 Proteins

The optimized SRorf279 and SRorf256 sequences were cloned via Golden Gate reactions between the constitutive Zea mays Ubiquitin promoter (proZmUbi, SEQ ID NO: 16) with the Zea mays ubiquitin intron (intZmUbi, exemplified in SEQ ID NO: 17) (Christensen, Sharrock, et Quail 1992) and a 3′ Sorghum bicolor Heat Shock protein (HSP) termination sequence (terSbHSP, depicted in SEQ ID NO: 18) (Putative uncharacterized protein Sb03g006880). The SRorf279 expression cassette depicted in SEQ ID NO: 24 and the SRorf256 expression cassette depicted in SEQ ID NO: 23 were separately cloned into the destination binary plasmid pBIOS10746. The binary destination vector pBIOS10746 is a derivative of the binary vector pMRT (WO2001018192).

Each binary plasmid described above was transformed into Agrobacterium EHA105. Each strain obtained was used for transforming BGA CMS*Fielder wheat cultivars as described in WO2000/063398. Wheat transgenic events were generated for each construct described above.

### 3—Fertility Restoration Phenotyping Assays

All wheat transgenic plants generated above and control fertile plants were grown in a glasshouse under standard wheat growth conditions (16 h of light period at 20° C. and 8 h of dark period at 15° C. with constant 60% humidity) until control grains of the wild type Fielder cultivar reached maturity stage.

Fertility of the transgenic plants was evaluated by counting the number of seeds and empty glumes per spikes on each plant and comparing with the wild type Fielder and BGA CMS*Fielder control plants. Plants are also evaluated by observing anther extrusion.

### D—Identification of Orf279

In order to identify orf279 in genomic DNA or RNA samples, the following primers can be used:
forward primers: SEQ ID NO: 12, SEQ ID NO: 6, SEQ ID NO: 10; and
reverse primers: SEQ ID NO: 7, SEQ ID NO: 11, SEQ ID NO: 9;
   or are constituted by the forward primer SEQ ID NO: 8 and the reverse primer SEQ ID NO: 9.

In order to identify the region in common with atp8 gene sequence, the following primers can be used:
forward primer: SEQ ID NO:12
reverse primer: SEQ ID NO:13

FIG. 5 shows the position of these marker sequences on orf279 genomic sequence.

### E—RFL29a Protein Optimization for Improving Suppression of Orf279 Expression

### 1—Designing and Obtaining Synthetic Optimized RFL29a Proteins

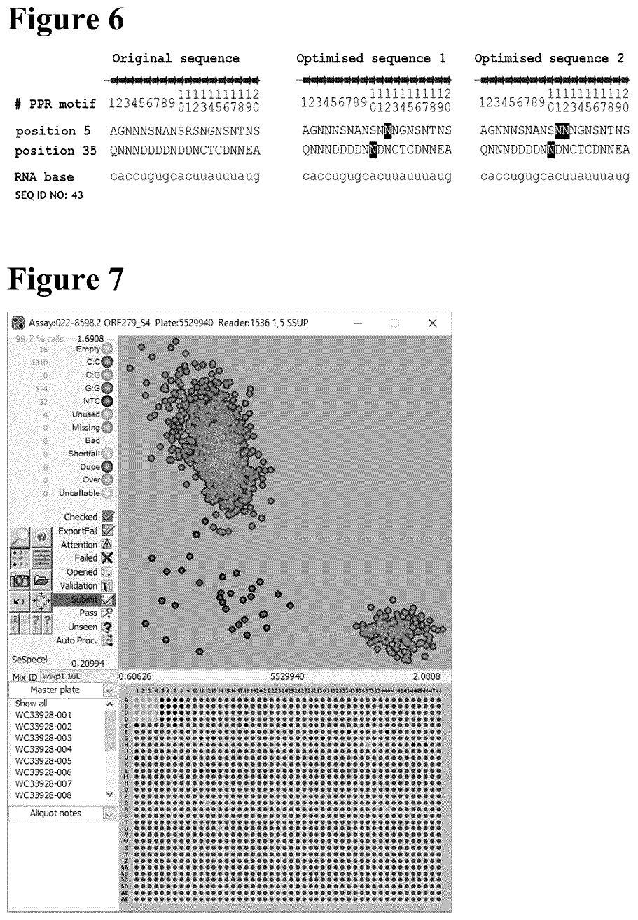

The RNA binding of RFL29a sequence within orf279 was analysed according to the PPR code described in Barkan et al. (2012) and patent application WO2013155555. The RFL29a sequence SEQ ID NO: 25 was optimized by altering the amino acid combinations at position 5 and 35 in the PPR motifs that are predicted (using −ΔG values calculated from Yan et al. 2019) to have weak affinity to the corresponding RNA bases (FIG. 6). Optimized RFL29a sequences are depicted in SEQ ID NO: 44 and SEQ ID NO: 45

### 2—Cloning and Transformation of Optimized RFL29a Proteins

The optimized Rf129a sequences SEQ ID NO: 46 and SEQ ID NO: 47 were separately cloned via Golden Gate reactions between the TaRFL29b promoter (pro TaRFL29b, SEQ ID NO: 48) and a TaRFL29a termination sequence (ter TaRFL29a, depicted in SEQ ID NO: 49. The optimised RFL29a expression cassettes respectively depicted in SEQ ID NO: 50 and SEQ ID NO: 51 were separately cloned into the destination binary plasmid pBIOS10746. The binary destination vector pBIOS10746 is a derivative of the binary vector pMRT (WO2001018192).

The binary plasmid described above was transformed into Agrobacterium EHA105. Each strain obtained was used for transforming BGA CMS*Fielder wheat cultivars as described in WO2000/063398. Wheat transgenic events were generated for each construct described above.

### F—Orf279 Diagnostic Marker for the Identification of Plants Harbouring a T-CMS Cytoplasm

A KASP design was developed to determine the presence or absence of the orf279 T-CMS in a plant material.

In order to determine if the orf279 T-CMS is present in a material, three primers were defined according to the PCR-based KASP technology:
the oligonucleotide AS1 (SEQ ID NO: 52) is specific from orf279 (sterile) and the genomic sequence from ChrUn ChineseSpring (IWGSC_V1).
the oligonucleotide AS2 (SEQ ID NO: 53) is specific from the normal cytoplasmic sequence (fertile).
the oligonucleotide C (SEQ ID NO: 54) is common between sequences.

The couple AS2/C is specific from the normal cytoplasmic sequence (fertile). The primer position has been optimized to exclude genomic paralogs amplification (more than 10 genomic paralogs copy from the fertile cytoplasmic sequence have been identified).

The couple AS1/C is specific of the orf279 T-CMS cytoplasmic sequence (sterile).

These three primers may be used simultaneously in a PCR amplification experiment (Kaspar protocol LGC Genomics) starting with genomic DNA (hybridization temperature=57° C.). End point fluorescence read, and clusters analysis of the samples reveal:
Vic fluorescence for sterile plants
Fam fluorescence for fertile plants

In FIG. 7, the cluster on the right side is the amplification of the normal cytoplasm with AS2 in fertile plants. The cluster on the left side is the amplification of the orf279 with AS1 in sterile plants (plants harbouring a T-CMS cytoplasm).

### BIBLIOGRAPHY

Kay R, et al. (1987). Duplication of CaMV 35S promoter sequences creates a strong enhancer for plant genes. Science 236:1299-1302.
McElroy D et al. (1990). Isolation of an Efficient Actin Promoter for Use in Rice Transformation. The Plant Cell, Vol. 2, 163-171.
Depigny-This D et al, 1992. The cruciferin gene family in radish. Plant Molecular Biology, 20:467-479.
Verdaguer et al. (1996). Isolation and expression in transgenic tobacco and rice plants, of the cassava vein mosaic virus (CVMV) promoter. Plant Molecular Biology 31:1129-1139.
Christensen A H and Quail P H (1996). Ubiquitin promoter-based vectors for high-level expression of selectable and/or screenable marker genes in monocotyledonous plants. Transgenic Res, May;5(3):213-8.
Götz H et al. (2011).Transgene Expression Systems in the Triticeae Cereals. Journal of Plant Physiology 168, no. 1:30-44. doi: 10.1016/j.jplph.2010.07.007.
Jones H D (2015). Wheat Biotechnology: Current Status and Future Prospects. K. Azhakanandam et al. (eds.), Recent Advancements in Gene Expression andEnabling Technologies in Crop Plants, DOI 10.1007/978-1-4939-2202-4_8.
Huang et al. (2009). Refining the Definition of Plant Mitochondrial Presequences through Analysis of Sorting Signals, N-Terminal Modifications, and Cleavage Motifs. Plant Physiology, July 2009, Vol. 150, pp 1272-1285.
Barkan A et al. 2012, PLosS Genet. A combinatorial amino acid code for RNA recognition by pentatricopeptide repeat proteins. 8(8):e1002910.
Christensen et al. (1992). Maize polyubiquitin genes: structure, thermal perturbation of expression and transcript splicing, and promoter activity following transfer to protoplasts by electroporation. Plant Mol Biol. 1992 February; 18(4):675-89.
Triboush et al. (1998), A Method for Isolation of Chloroplast DNA and Mitochondrial DNA from Sunflower, Plant Molecular Biology Reporter 16(2):183-183.
Ogihara et al. (2005). Structural dynamics of cereal mitochondrial genomes as revealed by complete nucleotide sequencing of the wheat mitochondrial genome. Nucleic Acids Res. 2005:6235-6250.
Song and Hedgcoth (1994A). A chimeric gene (orf256) is expressed as protein only in cytoplasmic male-sterile lines of wheat. Plant Mol Biol. 1994 October;26(1):535-9. Song and Hedgcoth (1994B). Influence of nuclear background on transcription of a chimeric gene orf256 and cox1 in fertile and cytoplasmic male sterile wheats.Genome, vol.37.
Small et al. (2004). Predotar: A tool for rapidly screening proteomes for N-terminal targeting sequences. Proteomics. 2004 June;4(6):1581-90.
Emanuelsson et al. (2007). Locating proteins in the cell using TargetP, SignalP and related tools. Nat Protoc. 2007;2(4):953-71.
1-2. (canceled)

3. A method for detecting orf279 DNA, orf279 RNA or Orf279 protein having an amino acid sequence with at least 95% identity to SEQ ID NO: 4, in a wheat plant, seed or bulk of seeds, the method comprising extracting a DNA or RNA or protein sample and detecting the orf279 DNA, orf279 RNA or Orf279 protein.

4. The method of claim 3, wherein the detecting of orf279 DNA, orf279 RNA or Orf279 protein comprises:
a. detecting the sterile cytoplasm with a marker at the recombination junction between atp8 and SEQ ID NO: 3, or within SEQ ID NO: 3, or
b. detecting the variation of orf279 expression in male sterile wheat plants.

5. The method of claim 3, wherein the orf279 DNA, orf279 RNA or Orf279 protein are detected by:
a. molecular markers and primers recognizing ORF279 recombination junction, or SEQ ID NO:3, or
b. antibody recognizing Orf279.

6. A method for detecting sterile plants harbouring a orf279 T-CMS cytoplasm or fertile plants harbouring a normal cytoplasm, the method comprising:
a) performing the method according to claim 3, wherein the DNA or RNA sample is extracted from the plants, and presence or absence of the orf279 DNA or orf279 RNA is detected by PCR amplification with suitable pair of primers to determine the presence or absence of orf279 T-CMS,
b) optionally, detecting the presence or absence of normal cytoplasm sequence by PCR amplification with suitable pair of primers, and
c) determining the fertile or sterile status of the plants.

7. The method according to claim 6, wherein the PCR amplification in step a) is performed using the pair of primers of SEQ ID NO:52 and 54, and optionally, the PCR amplification in step b) is performed using the pair of primers of SEQ ID NO:53 and 54.

8. A diagnostic marker for determining the presence or absence of orf279 comprising the pair of primers SEQ ID NO: 52 and 54 to amplify orf279 T-CMS sequence and, optionally, the pair of primers SEQ ID NO: 53 and 54 to amplify the normal cytoplasm sequence.


SEQUENCE LISTING (54 sequences)

SEQ ID NO: 1 - DNA, 837 bp, Triticum timopheevii
  source 1..837: mol_type = genomic DNA
atgcctcaacttgataaattaacttatttctcacaattcttctggttatgtcttctcctctttactttttatattctcttatttaataataataatggaatacttggaattagtagaattctcaaactacggaaccaactgctttcgcaccgggggggcgagatccggagcaaggaccctaagaatctggaagatatctcgagaaaaggttttagcaccggtctctcatatatgtactccagtttatccgaagtatcccaatggtgtaagaccgtcgactatttgggaaataagatatcttcttcaatctttctatactatcttaggggcgtcctttgcccaatttgcctcttattttttaaatttcttatttctttcgccttcacctgtgcacttatttatgtattcaagggggggggtttcgtggctatggctgctacaaacggagcctcttcttcctttctggagagctcaggtgaaatagatgcactgttacaaacaacaacaacaacacgaacacccgaaaacgcggattacgaagacgataatacttctgtaaatcaagaattcctcgaagacggacaaagggcccgacaggctaaactacgggagttagaaagactcattctccagcaatataaggatttcatccgacagaaatatccatggatacctaaaggcgacatccttctcccgagcatgaagggtggagttgtcgaggacgtaatggaaaaattagaattggaaacgtattccagtagcgacttgactgattggatcaaccatttgcgggcaaatccgaaaacattaaattttatcttcaaggattttgtcgcg

SEQ ID NO: 2 - DNA, 290 bp, Triticum timopheevii
  source 1..290: mol_type = genomic DNA
atgcctcaacttgataaattaacttatttctcacaattcttctggttatgtcttctcctctttactttttatattctcttatttaataataataatggaatacttggaattagtagaattctcaaactacggaaccaactgctttcgcaccgggggggcgagatccggagcaaggaccctaagaatctggaagatatctcgagaaaaggttttagcaccggtctctcatatatgtactccagtttatccgaagtatcccaatggtgtaagaccgtcgactatttgggaaa

SEQ ID NO: 3 - DNA, 547 bp, Triticum timopheevii
  source 1..547: mol_type = genomic DNA
taagatatcttcttcaatctttctatactatcttaggggcgtcctttgcccaatttgcctcttattttttaaatttcttatttctttcgccttcacctgtgcacttatttatgtattcaagggggggggtttcgtggctatggctgctacaaacggagcctcttcttcctttctggagagctcaggtgaaatagatgcactgttacaaacaacaacaacaacacgaacacccgaaaacgcggattacgaagacgataatacttctgtaaatcaagaattcctcgaagacggacaaagggcccgacaggctaaactacgggagttagaaagactcattctccagcaatataaggatttcatccgacagaaatatccatggatacctaaaggcgacatccttctcccgagcatgaagggtggagttgtcgaggacgtaatggaaaaattagaattggaaacgtattccagtagcgacttgactgattggatcaaccatttgcgggcaaatccgaaaacattaaattttatcttcaaggattttgtcgcg

SEQ ID NO: 4 - protein, 279 aa, Triticum timopheevii
MPQLDKLTYFSQFFWLCLLLFTFYILLFNNNNGILGISRILKLRNQLLSHRGGEIRSKDPKNLEDISRKGFSTGLSYMYSSLSEVSQWCKTVDYLGNKISSSIFLYYLRGVLCPICLLFFKFLISFAFTCALIYVFKGGGFVAMAATNGASSSFLESSGEIDALLQTTTTTRTPENADYEDDNTSVNQEFLEDGQRARQAKLRELERLILQQYKDFIRQKYPWIPKGDILLPSMKGGVVEDVMEKLELETYSSSDLTDWINHLRANPKTLNFIFKDFVA

SEQ ID NO: 5 - protein, 183 aa, Triticum timopheevii
NKISSSIFLYYLRGVLCPICLLFFKFLISFAFTCALIYVFKGGGFVAMAATNGASSSFLESSGEIDALLQTTTTTRTPENADYEDDNTSVNQEFLEDGQRARQAKLRELERLILQQYKDFIRQKYPWIPKGDILLPSMKGGVVEDVMEKLELETYSSSDLTDWINHLRANPKTLNFIFKDFVA

SEQ ID NO: 6 - DNA, 17 bp, synthetic construct
  misc_feature 1..17: note = primer PP_03004_ORF279-amont-cliv_3_F
  source 1..17: mol_type = other DNA
ttcgccttcacctgtgc

SEQ ID NO: 7 - DNA, 20 bp, synthetic construct
  misc_feature 1..20: note = primer PP_03004_ORF279-amont-cliv_3_R
  source 1..20: mol_type = other DNA
cctgagctctccagaaagga

SEQ ID NO: 8 - DNA, 20 bp, synthetic construct
  misc_feature 1..20: note = primer PP_03006_ORF279-aval-cliv_4_F
  source 1..20: mol_type = other DNA
aattcctcgaagacggacaa

SEQ ID NO: 9 - DNA, 19 bp, synthetic construct
  misc_feature 1..19: note = primer PP_03006_ORF279-aval-cliv_4_R
  source 1..19: mol_type = other DNA
caactccacccttcatgct

SEQ ID NO: 10 - DNA, 17 bp, synthetic construct
  misc_feature 1..17: note = primer PP_03007_ORF279-cliv_F
  source 1..17: mol_type = other DNA
gggggtttcgtggctat

SEQ ID NO: 11 - DNA, 19 bp, synthetic construct
  misc_feature 1..19: note = primer PP_03007_ORF279-cliv_R
  source 1..19: mol_type = other DNA
cttcgtaatccgcgttttc

SEQ ID NO: 12 - DNA, 20 bp, synthetic construct
  misc_feature 1..20: note = primer PP_03003_atp8_4_F
  source 1..20: mol_type = other DNA
caaactacggaaccaactgc

SEQ ID NO: 13 - DNA, 21 bp, synthetic construct
  misc_feature 1..21: note = primer PP_03003_atp8_4_R
  source 1..21: mol_type = other DNA
ccattgggatacttcggataa

SEQ ID NO: 14 - DNA, 1992 bp, synthetic construct
  misc_feature 1..1992: note = proZmUBI_intZmUBI
  source 1..1992: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 15 - protein, 792 aa, Triticum aestivum
MPRFSSTTPMSPPRLLLRLGARHSSSTSHPSRIWDPHAAFAAATQRARSGTLTTEDAHHLFDELLRQGNPVQERPLTNFLAALARAPASAFCSDGPALAVALFGRLSRGAGRRVAQPNVFTYGVLMDCCCRARRLDLAIAFFARLLKTGLEANQVIFCTLLKGLCHAKRSDEALDVVLHRMPELGCTPNVVAYTTVIHGFLKEGQVGKACNLFHGMAQQGVAPDVVTYNSVIDALCKARAMDKAEYFLREMVDNGVVPNNVTYNSLIHGYSSLGHQKEAVRVLKEMTRQGIIPDVITCTSLMTFLCKNGKSKEAAEIFDSMATKGLKHDAVSYAILLHGYATEGCLVDMINLFNSMDRDCILPNCHIFNILIYAYAKSGKLDKAMLIFRDMQKQGVSPDAFTYSTLIHAFCKKGRLDDAMIKFNQMVDTGVRQGTAVYGSLIQGFCTHGDLVKGKELVTEMMNKGIPPPDIMFFHSIMQNLCTEGRVVEARDILGLIAHIGMRPNVCTFNILIGGYCLVRKMEDASKIFHDMMSYGLEPSNVTYGILINGYCKNRRIDDGLILFKEMLRKGLKPTTFNYNIILDGLFLAGRTVAAKEKFDEMVESGVSMCISTYSIVLRGLCRNNCSGEAITLFQTLSAMDVKFNIRIVNIMIDAFFRVQRKQEAKDLFAAITANGLVANVFTYSLMMTNLIKEGSVEEADTLFLSMEMSGCTSNSWMLNLIIRGLLEKGEIVKAGCYMSKVDAKSYSLEAKTVSLLIYLFSGKGKYREHIRLLPTKYQFLEEAATVEWFAI

SEQ ID NO: 16 - DNA, 978 bp, Zea mays
  source 1..978: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaag

SEQ ID NO: 17 - DNA, 1014 bp, Zea mays
  source 1..1014: mol_type = genomic DNA
gtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 18 - DNA, 450 bp, Sorghum bicolor
  source 1..450: mol_type = genomic DNA
gcatccatggacgtggattgaattgaaggtgtactactgctgtgctggtccgtggatcgtggctgtcatgcatggtttgctgtgtcttctacgatatgtacttccctttgttccgtatatgtacatcttcctcgtttggttcatgtattttcctttgaataataataaataaatcgggctttccatatcggatgcttttatatctgtgtgtatggagattgtggtatatggtttcatctcaagttgtttacgtcaagaactaaagatatttcctcaaaaaaaaagaactaaagatataatcaatgtcattaacataactcatttccatgaggagaggacgaaggacgaagtcataataagtagattggttgatattttataatcattcaaaactgcaggggttataagatcttcattttgtagaagttttagatcttccgaggggttctc

SEQ ID NO: 19 - protein, 43 aa, Oryza sativa
MSRRHHLHLPLRLLSRNNPSAPLFRHAFSTLDTPEPPPPETEA

SEQ ID NO: 20 - protein, 66 aa, Oryza sativa
MATRRALTSVLRSASRLRAASPSPCPRRAPLHPHRRPSPAGFLLNRAAAAYASSAAAQAAPAPPPA

SEQ ID NO: 21 - protein, 829 aa, synthetic construct
  REGION 1..829: note = SR-256
MSRLRLPRGSSSSTTLIPRLRLLRRCSTFTSTSSPSRSWSPRDAFAAATERIRAGTLSPEDAHKLFDELLGKATPVPERSLNGFLAALARAPASGNCIRDGPALAVALFNRVCREEAGPQVAALTVCTYNILMDCCCRARRPDIGLAVFGRFLRKGLKTDQTGANTFLKCLCYAKRTDEAVNVLLHRMSELGCVPNAISYNTVLKGLCDNSMSQRALDLLQMVARKGGGCFPDVVAYSTVIHGFFKEGETGKACSLFHEMMQQGIVPSVATYNSIIDALCKVRAVDNAELVLRQMVAKGAQPDTVTYNCMINGYATSGRLKEAAKMFREMKSRGLTPNVVTCNSFLASLCKHGTSKEAAEFFDSMTAKGQKPDIISYCTLLRGYASEGCFTDMIDLFNSMKSNGIAADCHVFTILIDTYAKRGMMDDAMHIFTEMRQQGVSPNVVTYSTVISTLSRMGRLTDAMEKFNQMVALGVQPDRAIYNSLIQGFCMHGGLVKAKELVSQMINKGIPHPNIVFFNSVINSMCKEGRVMDAHDILDLVIDIGDRPNDISFNSLIDGYCLVGKMDKAFGMLNAMESVGVEPDIVTYNTLVKGYCRNGRIDDGLTLFREMLCKGVKPDTVTYNIVLNGLFHSGRTVAARKMFHQMIESGTTVNISTYGIILGGLCRNNCADEAIALFQKLGAMNVKFSITILNTMINAMYKVQRKEEAKELFGTISASGLVPNESTYGVMIKNLLEEGSVEEADNMFSFMDRSGIVPDSRLMNDIIRMLLEKGEIAKAGYYLSKVDGKSISLEASTTSLMFSLFSRKGTYKEDMKLLPAKYQFFGGVG

SEQ ID NO: 22 - protein, 813 aa, synthetic construct
  REGION 1..813: note = SR-279
MSGVCLRRRLSSTSTSNTPPSPSWSPQAAFAAATERARAGTLSPQDAHHLFDELFRQATPVPERSLEGFLAALGRAASSEACRDGPSLALTLFNRLCREEAGRRVAPPTIYTYNILMNCCCRVRRPDLGLAFFGRLLRTGLKTNEVVASTLLKCLCCAKRADEAVNVLLHRMSVLGCVPNSFSYNIVLKSLCDDSRSQRALGLLQMMAKGGVCSPNVLSYNTVIHGFFKEGEIDKACNLFHEMTKQGFVPNVVTYNSVINALCKARAMDNAESFLRQMVDNGVPPDKVTYTSIIHGYSTLGRWKEATKMFREMTSRGLIPDIVTRNSFMDSLCKHGRSKEAAEIFFSMAARGHKPDIVSYTILLHGYGNEGSFSDMMSLFNSMEGGGIVADCQVFNILIDAYAKRGMIDEAMLIFTEMLGQGVNPSVVTYSTLIAALCRTGRLADAMDKFSQMVGTGVQPNTVVYHSLIQGFCTHGDLVKAKELISEMMNKGIICPNIAFFNSIVDSLCKEGRVMDAHHIFDLVKDIGEKPDAIMFNTLIDGYCLVGEMGKAFMVLDVMTSAGIEPDAITYSTLVSGYCKSGRIDDGLILFREEMLHKKVKPTIVTYNIILEGLFRAGRTVAAKKMLHEIIGSGTPVDMHTYNIFLRGLCRNNCADEAIVLFQKSRAMNVKFNITTLNTMINAFYKGDRREEANDLFAALPASGLVPDASTYGVMIQNLLKEGAVEEADNMFSSMEKSGCAPSSRLVNDVIRTLLEKGEIVKAGKYMSKVYGKSLSLDASTSSLLLSLFSGNGKYREQIQLLPAKYQFFGGIS

SEQ ID NO: 23 - DNA, 5010 bp, synthetic construct
  misc_feature 1..5010: note = proZmUBI_intZmUBI_SR_256_terSbHSP
  source 1..5010: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatgtctcgcctccgcctcccccgcggcagctcctcctccaccacgctgatcccccgcctccggctcctccgccgctgctccaccttcacctccacctcgtcgccctcacgctcctggtctccccgagacgcctttgccgcggccacggagcgtattcgcgccgggacgctcagcccggaagacgctcacaaactgttcgacgaattgctcggtaaggccaccccggtcccggagcgctccctgaacggcttccttgccgccctcgcccgtgcgcccgcctccggcaactgcatcagagatggccccgccctcgcagtggctctcttcaaccgtgtgtgcagagaggaagccggcccgcaggtggcggcgctcacagtttgcacctacaacatcctcatggactgctgctgccgtgcgcgtcgtccggacatagggcttgccgtatttggccgcttcctcaggaagggcctgaagacagaccagaccggcgccaacaccttcctcaagtgcctctgctacgcgaaacggacggatgaggctgtgaacgtgctgctccacaggatgtccgagctcggctgtgtgcctaatgccatttcgtacaacactgttctgaagggcttatgcgacaatagcatgagtcagcgggcgctcgacctgctccagatggtggcgagaaaaggaggtggctgcttccctgatgtggtggcgtatagcacggtcatccatggcttttttaaggagggtgaaacagggaaggcatgcagtctattccatgaaatgatgcaacaagggattgtgcccagtgtggcgacatataactcgattattgatgcgttgtgcaaggtcagagcagtggacaatgcagagctagtccttcgtcagatggttgctaaaggtgctcaacccgatacagtgacatataattgcatgatcaatggatatgccacatctgggcggttgaaagaggctgctaaaatgttcagagaaatgaaaagccggggtcttacaccaaatgttgttacttgcaactcgttcctggcctccctttgcaagcacggaacaagcaaagaagctgcagaattttttgattctatgacagccaagggccaaaaacctgatatcatctcgtactgtactttgcttcgtgggtatgccagtgaaggatgctttactgatatgattgatctctttaattcaatgaaaagcaatggtattgcagctgactgccatgttttcaccatattaattgatacctatgctaaacgcggaatgatggatgatgcaatgcacatatttacggaaatgcggcaacaaggtgtgagtccaaatgttgtcacatattcaactgtaatatcaacactttctagaatgggtaggttgaccgatgctatggaaaaatttaatcagatggttgccttgggagtacagccagatagagctatttataactccctaattcagggattttgtatgcatggtggtttggttaaagctaaggagttggtttctcaaatgataaacaaaggtattcctcatcctaacattgtgttcttcaattcagtaataaacagtatgtgcaaggaaggcagggttatggatgcacatgatatccttgacttggttatagacataggtgataggcctaatgacatttcatttaattcactgattgatggatattgcttagtcgggaagatggataaagcattcggaatgcttaatgccatggaatcagttggtgttgagcctgatattgttacttacaatacacttgttaagggctattgtagaaatggaaggatcgatgatggtttaactctgtttagagaaatgttgtgcaagggagttaaacctgacactgtgacatataacatcgtactgaatgggttgtttcattctgggagaactgttgctgcaaggaaaatgttccatcagatgattgaaagtggaacaacagtgaacatttccacatacggtataatacttggaggtctttgtagaaataattgtgcagacgaagcgattgccctgttccagaaattaggagcaatgaatgtaaagttcagtattacaatactcaataccatgattaatgcaatgtacaaggttcaaagaaaagaagaagctaaggagttgttcggtacaatatcagccagtgggttggtgcctaatgaatctacttatggagtaatgataaaaaatcttctcgaagaaggatcagtggaagaagctgacaatatgttttcgtttatggacaggagtggtatagtccccgactcccgtctcatgaatgatatcatcagaatgttgttggaaaaaggtgagatcgccaaggccggatattatctgtctaaagttgatggcaagagcatatcacttgaagcttcaactacttcattgatgttttctctcttctcaaggaaaggcacatataaggaggacatgaaattgctccctgcaaagtatcaatttttcggtggagttggtgattacaaggatcacgacggggactacaaagaccatgatattgactacaaagacgatgacgacaaatgaaacgcatccatggacgtggattgaattgaaggtgtactactgctgtgctggtccgtggatcgtggctgtcatgcatggtttgctgtgtcttctacgatatgtacttccctttgttccgtatatgtacatcttcctcgtttggttcatgtattttcctttgaataataataaataaatcgggctttccatatcggatgcttttatatctgtgtgtatggagattgtggtatatggtttcatctcaagttgtttacgtcaagaactaaagatatttcctcaaaaaaaaagaactaaagatataatcaatgtcattaacataactcatttccatgaggagaggacgaaggacgaagtcataataagtagattggttgatattttataatcattcaaaactgcaggggttataagatcttcattttgtagaagttttagatcttccgaggggttctc

SEQ ID NO: 24 - DNA, 4962 bp, synthetic construct
  misc_feature 1..4962: note = proZmUBI_intZmUBI_SR_279_terSbHSP
  source 1..4962: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatgtccggcgtctgcctccgccgccgcctctcctccacctccacctccaacacgccaccctcaccttcctggtcaccccaggccgcctttgccgcggccacggagcgcgcccgcgccggaacgctcagccctcaagacgcacaccacctgttcgacgaattgtttcggcaggccactccggttcccgagcgctcccttgaaggattccttgccgccctcggccgtgctgcatcctctgaagcctgcagagacggcccctcccttgccctcactctcttcaaccgtctctgccgagaagaagccggccggcgggtggcgccgcccacaatctacacctacaatatcctgatgaactgctgctgccgcgtgcgtcgtccagacctagggctcgccttcttcggccgcctcctaaggaccggcctcaaaacaaatgaggtcgtcgccagcaccctcctcaagtgcctctgctgtgcaaaacgggcagatgaagctgtgaacgtgctgcttcataggatgtccgtcctcggctgtgtgcctaattccttctcatacaacatagttctaaagagcttatgtgatgacagcaggagccagcgagcgctcggcctgctccagatgatggcaaaaggaggtgtctgctcccccaacgttctgtcatataacacagtcatccacggcttctttaaggaaggtgaaatagacaaggcctgcaatctattccatgaaatgacgaagcaagggtttgtgcctaatgtggtgacatataactcggttatcaatgcattgtgcaaggcccgagcaatggacaatgcagagtcgttccttcggcagatggttgataacggtgttccacctgataaggtgacatatactagcatcatccatggatattccactttgggccgatggaaagaggcaactaaaatgttcagagaaatgacaagcaggggtcttataccagatattgtcactcggaactcattcatggactcactctgcaagcatggaagaagcaaagaagctgcagaaattttcttttccatggctgcaaggggccacaagcctgatatcgtctcctacactattcttctccatgggtatggcaatgaaggaagcttttctgatatgatgagtctctttaattcaatggaaggcggcggtattgtggccgattgccaagttttcaacatattaattgatgcatatgctaaacgaggaatgatcgacgaagctatgctcatatttactgaaatgctaggacaaggagtcaatccgagtgtagtcacctactcaactctgatagctgcactttgcagaacaggtaggctagctgatgctatggataagtttagtcagatggttggtacgggagtacaaccgaacacagttgtttatcactccctaattcagggtttctgtacacatggcgatttggtcaaagcgaaggaattgatttctgaaatgatgaataaaggtattatttgtccaaacattgcattcttcaattcaatagtagacagtctatgcaaagaaggaagggttatggatgcacaccatatctttgacttggttaaagacataggtgagaaacctgatgccatcatgtttaatacgctaattgacggatattgcttagttggtgagatgggcaaagcattcatggtacttgatgtcatgacatcagctggcattgagcctgatgccattacgtatagtacacttgtcagtggatattgtaaaagtggaaggatcgatgatgggttgattctgttcagagaagaaatgttgcataagaaagttaaaccaacaattgttacatataacatcatattggaggggttatttcgtgctgggagaacagttgctgcaaagaaaatgctccatgaaattatcggaagtgggacacctgtggacatgcatacatacaacatatttcttagaggactttgtagaaataattgcgcggatgaagcgatcgtcctattccagaaatcacgtgcaatgaatgtgaagttcaatatcacaacactcaataccatgattaatgcattctacaagggtgacagaagagaagaagctaatgatttgtttgctgcattaccagccagcgggttggtgcctgatgcttccacttacggagtaatgatacaaaaccttctaaaagaaggagcagtggaggaagctgacaatatgttctcatcaatggagaagagtggttgtgctcctagctctcgtcttgtgaatgatgtcatcagaactttattggaaaagggggagatagtcaaggccggaaaatatatgtctaaagtttatggcaagagcctatcacttgatgcttcaactagttcgttgttgttgtctctcttttcagggaatgggaaatatcgggaacaaatacaattgctccctgcaaagtaccagtttttcggtggaatcagtgattacaaggatcacgacggggactacaaagaccatgatattgactacaaagacgatgacgacaaatgaaacgcatccatggacgtggattgaattgaaggtgtactactgctgtgctggtccgtggatcgtggctgtcatgcatggtttgctgtgtcttctacgatatgtacttccctttgttccgtatatgtacatcttcctcgtttggttcatgtattttcctttgaataataataaataaatcgggctttccatatcggatgcttttatatctgtgtgtatggagattgtggtatatggtttcatctcaagttgtttacgtcaagaactaaagatatttcctcaaaaaaaaagaactaaagatataatcaatgtcattaacataactcatttccatgaggagaggacgaaggacgaagtcataataagtagattggttgatattttataatcattcaaaactgcaggggttataagatcttcattttgtagaagttttagatcttccgaggggttctc

SEQ ID NO: 25 - protein, 788 aa, Triticum aestivum
MPRFSSTTPMSPPRLRLRLCARHSSSTSHPSRIWDPHAAFAAAAQRASSGTLTTEDAHHLFDELLRRGNPVQERPLNKFLAALARAPASASCCDGPALAVALFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAILLHGYAAEGCLVDMINLFNSMERDCILPDCRIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 26 - DNA, 2178 bp, Triticum aestivum
  source 1..2178: mol_type = genomic DNA
gtaggatgtgtgcggggtgattggaagatccaactataaatattattgatactctcttaaaagatgggatggtgatcaccttgcaataaagagttattttttcgcgaatagcaataaagagttatttgatggcaaaaaaaaatcagatgatgtgggctggcctaggatatatagatatataacacttccccttttatattagaatacttaaacatttcgtacattgcatgttgttatatagcctgcaaaaaaggctttcgcgctgctttatataaaaagcaacatagtcaaactgatgtatggtgatgagaagatcgtcttataaaacagatcatatggaaaaacaagaaaacaatagagtgcacaccctatcacaaaacaatctagactacaactagggggagacttgcaccaagggaaaacgcatgacctcgttgcgcaactcgtaccaaggtgacaccagagagagcccctaccctcccgctcctgatcgtggagcctaagcctgcttgcggacaacaaggatcgcgatggatgccaataagggctcgtgtaaaaagataaggaggaaaaacgacagcccacgcgcgaagccaaggaaacttcgatccagcatccgaacatgcctgccaattggcgacacggtgcaccgaaggtaacctaggccccatgacgacgcccacaggagggagacgacgtaaggagtcgccatcgtcaaaaccgaagagacaatcaagggttttcatccggagccctggctccaagagagggaccacatcgacggccccaagagagatgcgacacccatgtgcatcactgccactagtgtcgaagcacaaggctttcgcctagagcctcttacaggcgtcacatcgaggaaccccgagcatcagcccacccgagggaagagatatcactaccaaacaaggcctccatggttctgggagccgtcactgccgaggcaccaccgacaccgggatcaccaagcgtggcatcctttgccatccacacaaccaaaagatgtccccaccaccgctgccaaggcgcctgctgaaaagccgctctgacggtccgccaccctaggccgccgctccggcgtccaatatcccaacctcgactccacacgcgggcaccaacccccgaaaaccgcactcaccggtggtgacacggaaagcgaaactactccatgcacgacgttcagtgaacgatctctggacgacaaggaatcaaatggctggagctgtactttgtctattcatgatcggtgtgatcaatagcgtcgtccacagatcgggcaaaggccgtcgtccgcataacatcgctccatggaaaggctcttcctttccagtcccacggcacaccacgccgacaccagcaccatctgcggcggtgaccactgaccagcgaacctaactcggtcaaggagaccaaacctggctgggagcacgctaccccatctgcctccaccccacagcacgagcacgggtgtccattcaggtacaacaggggatccacatcagaacatctccaacaatttgtatgttagtttgttggtaaaatatgccatgtcatcaaccaacaccgtaacatacaacaacttcaatggattatatctagcctgcccaatagaatgtgagataataaataaggtgctctctcattctacattggagcttgtgcaaggagtgttgattcatgtacatacaaccttcttctctttcttcatttattgtatgacacatcatcaaaaattctactacaccctttccgatttataaggcatgcacgtatccctaggtttataatttgagcaatataaaataagttgtatattctaaaaattacaccattagaaagtagaaaatctgaaatttttaataatatattttttgtgacaaatagcttgttttacgttggtttatttgtcaacctagatatacgtgcaagccctataaatcggaaaggaggtagtatgtgacaaagtctaccaagaaatatcttataactattgagatgcccttagcgagagctggctgccgattgactgatgaatgaacaaaaatcctatatgacaaagtctaccaagaactattttataactattgccgattgactgatgaatgaaccgttaccgtcttcttgtacagaagtaaacacttgtatacctccaccgccccg

SEQ ID NO: 27 - DNA, 1716 bp, Triticum aestivum
  source 1..1716: mol_type = genomic DNA
catatttccttcactagcttggtgcgaggcttgcaactttctcctgtgatgctcatcaacaaaatcggagctgtcggtcctcgacgcctgcggccgatcgtttggcttggctgtcgaggtcatcgggtgtcgtttgttcagaggggtcttgatgggtcgtcagcgaggaaaccagagttgcctgctcgcagagaggcgatgacgatgacattgcttgcagcctcgacggtgtcctctcctcagcggtatgtgtgtggtcttgtcagtgccaggtcggccggagcgccctattcgatgggcgtgttttaattaaccgagcaactctcttcttcttaatcaatgaaaatggcaagtcttttaccttgtttcaaaaagagttagatgcactggaggtcctaaaggtttcgtcgaggtgtcagtttagtcctatgtcttccaaaatgcgcctttaggtcctaattctttcttaagtggttcacccgaggtccttttccacgaccactcgctgaccagcccacgtgccgcattgacccacgccacgtcgacagagggtccaaccgccaggcgagaagcttcgcaaagagacaccccccctcttagtggcacccctctcctctttcacggtactttggcgacattggaggcgagtggcagaggcaaccggaggtgatctggaggcaatctgagtgggtgcccgtgaagatgccccttctacgacggccagtgagcgatcctcctccatgttaccgtgagaaccctaatcttgtcgatgctcttctcctgtagctcttctaatatgccataactctgccataatcaatagttgtagtccttgatttactttgatttgcataacggtgctacggacacggcaaaaaggacacaccaccggcgacaacatacccgccgaagttggagcttgcttcgccgcgccggtggcggtgctgacgatgctgccatgctcctcatcttcagttgtttccttgccgaggagcttgaagttccggccatggataggtttagggcacacgagagagcagatttggggaagaaataaagtcagggactggggaatgagaaggtaatgttcataaggggttttctgtaaaaagaaagagcaacctaagcgacccctttgtcgacttggcgtgggtcaatgcgccacacaggcaggtcaatgagcgcttgtgagaaaaaggtcctcgggtaaaccacttgcttaaagaattactccctccgtcccatattataagaacgtttttgacactagtgtagtgtcaaaaacgttcttatattatgggacggagggagtagtacctaaaggtgcattttggactaaataagactaaagtgacacccagacgaaacttttaggacctcgagtgcatctaactcttaaaaaaacaaagatgctaaaaaaactaaatgcttaaaaatatatttttctatgggaatacttaaaaatactattataattttatggcttcctagtatatttctagggcttacaactaaatttatattcttggtctaaataaaattataatctgataaattatgtgatatgaatatgtcatataggcatatgagaccataatttaattttacctgcaaaaaataagtaatagtagtagtactactagataagataacttggtaaagcactagccccgtcgtctcccagttcaggatctacctacactgcacaccaaggcc

SEQ ID NO: 28 - DNA, 2500 bp, Triticum aestivum
  source 1..2500: mol_type = genomic DNA
ggtggcggcccccctttcctgcctttggtcattcgtctccgcgcaggtcggctgctctggccggcccttggtggttgctgtagtgctgcggcaagggggtgccagatccatcggttcttcattcggatccagctcgtctcttgccttactcagggtcggtcaccccgtgaccttgcctgcggcagtgagggggtgtgcgtgctctgtgatgggccttgtgaggcgatggcgcgcttgctgcgcaggggtgtgcgggccaacttgtgtcggctgcgtgagggtgttggttgagtctgctccaagcgcaagcttgcccggctatggccggccggtggtggcggcgcctacgggtgtcgtttcccttgttgaaggcgttgttgtggagttcttcgacctactacaatctcgaatctggttcttcgggcgaaagccttgccccatctgggtcgggcaacgacgtcgtccgtacgatgttttccccctttggggcgttgctttgaaggccggttaacctttcccgtgctacctttgggttggtcgtgcatggtgtcatggttggctggtgctcaaccggtgatgcgagtggttggcgttgcgactcgtgcggcgacgaagatggtgatgtgcagagctgggtcgcggcttgtccttctgatgtcggcggttcgattcccctttgggtgcatctgtgcttgtttctcactatgacagagaagttgaagctgcatgcagcggggcccttgcagcaatgatgactccatgagggtggtttcggcagacggtgctctccgtaggttgcactggactagcttggtgcgaggcttgcaactttctcctgtgatgctcatcaacaaaatcggagctgtcggtcctcgacgcctgcggccgatcatttggcttggctgtcgaggtcatcgggtgtcgtttgttcagaggggtcttgatgggtcgtcagcgaggaaaccagagttgcctgctcgcggagaggcgatgacgatgacattgcttgcagcctcgacggtgtcctctcctcagcggtatgtgtgtggtcttgtcagtgccaggtcggccggagcgtcctattcgatgggcgtgttttaattaaccgagcaactctcttcttcttaatcaatgaaaatggcaagtcttttacctcgtttcaaaaagagttagatgcactggaggtatcgagatgtcagtttagtcctatgtcttccaaaatgcgcctttaggtcctaattctttcttaattggttcacccgaggtccttttccacgaccactcgctgaccagcccacgtgccgcattgacccacgccacgtcgacagagggtccaaccgccaggcgagaagcttcgcaaagagacaccccccctcttagtggcacccctctcctctttcacggtactttggcgacattggaggcgagtggcagaggcaaccggaggtgatctggaggcaatctgagtgggtgcccgtgaagatgccccttctatgacggccagtgagcggtcctcctccatgttaccgtgagaaccctaatcttgtcgatgctcttctcctgtagctcttctattatgccataactctgccataatcaatagttgtagtccttgatttacttcgatttgcataacggtgctacggacacggcaaaaaagacacaccaccagcggcaacatactcgccgaagttggagcttgcttcgccgcgccggtggcggtgctgacgatgctgccatgctcctcatcttcagttgtttccttgccgaggagcttgaagttccggccatggataggtttagggcacacgagagagcagatttggggaagaaataaagccagggactggggaatgagaaggtaatgttcataaggggttttctgtaaaaagaaagagcaacctaagcggcccctttgtcgacttggcgtgggtcaatgcgccacataggcaggtcaatgagcgcttgtgagaaaaaggtcctcgggtaaaccacttgcttaaagaattactccctccgtcctataatataagaacgtttttgacactcactagtgtagtgtcaaaaacgtttttatattatgggacggagggagtagtacctaaaggtgcattttggactaaataagactaaagtgacaccccgacgaaacttttaggacctcgagtgcatttaactcttaaaaaaacaaagatgctaaaaaaactaaatgcttaaaaatatatttttctatgggaatacttaaaaatactattataattttatggcttcctagtatatttctagggcttacaactaaatttatattcttggtctaaataaaattataatctgataaattatgtgatatgaatatgtcatatagccatatgagaccataatttaattttacctgcaaaaaataagtaatagtagtagtactactagataagataacttggtaaagcactagccccgtcgtctcccagttcaggatctacctacactgcacaccaaggcc

SEQ ID NO: 29 - DNA, 4833 bp, synthetic construct
  misc_feature 1..4833: note = proZmUBI_intZmUBI_TaRFL79_terSbHSP
  source 1..4833: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgccaagagctactcacttgaagctaaaactgtttcgttgctgatctatctcttttcagggaaagggaaatacagagaacacataagattgctacctacaaagtatcagtttctcgaagaagcagccacagttgaatggtttgctatatagaacgcatccatggacgtggattgaattgaaggtgtactactgctgtgctggtccgtggatcgtggctgtcatgcatggtttgctgtgtcttctacgatatgtacttccctttgttccgtatatgtacatcttcctcgtttggttcatgtattttcctttgaataataataaataaatcgggctttccatatcggatgcttttatatctgtgtgtatggagattgtggtatatggtttcatctcaagttgtttacgtcaagaactaaagatatttcctcaaaaaaaaagaactaaagatataatcaatgtcattaacataactcatttccatgaggagaggacgaaggacgaagtcataataagtagattggttgatattttataatcattcaaaactgcaggggttataagatcttcattttgtagaagttttagatcttccgaggggttctc

SEQ ID NO: 30 - DNA, 4821 bp, synthetic construct
  misc_feature 1..4821: note = proZmUBI_intZmUBI_TaRFL29a_terSbHSP
  source 1..4821: mol_type = other DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtctccaccatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagtggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagaacgcatccatggacgtggattgaattgaaggtgtactactgctgtgctggtccgtggatcgtggctgtcatgcatggtttgctgtgtcttctacgatatgtacttccctttgttccgtatatgtacatcttcctcgtttggttcatgtattttcctttgaataataataaataaatcgggctttccatatcggatgcttttatatctgtgtgtatggagattgtggtatatggtttcatctcaagttgtttacgtcaagaactaaagatatttcctcaaaaaaaaagaactaaagatataatcaatgtcattaacataactcatttccatgaggagaggacgaaggacgaagtcataataagtagattggttgatattttataatcattcaaaactgcaggggttataagatcttcattttgtagaagttttagatcttccgaggggttctc

SEQ ID NO: 31 - DNA, 24 bp, synthetic construct
  misc_feature 1..24: note = primer GSP1
  source 1..24: mol_type = other DNA
ggatttgcccgcaaatggttgatc

SEQ ID NO: 32 - DNA, 47 bp, synthetic construct
  misc_feature 1..47: note = primer GSP2
  source 1..47: mol_type = other DNA
gattacgccaagcttaagaatcagaattactgagctaccccgctctt

SEQ ID NO: 33 - DNA, 22 bp, synthetic construct
  misc_feature 1..22: note = primer P1
  source 1..22: mol_type = other DNA
atgacaaatatggttcgatggc

SEQ ID NO: 34 - DNA, 18 bp, synthetic construct
  misc_feature 1..18: note = primer P2
  source 1..18: mol_type = other DNA
gcttggggatcctgaatc

SEQ ID NO: 35 - DNA, 19 bp, synthetic construct
  misc_feature 1..19: note = primer P3
  source 1..19: mol_type = other DNA
gctgtcactagaacggacc

SEQ ID NO: 36 - DNA, 22 bp, synthetic construct
  misc_feature 1..22: note = primer Ta_Actin_F
  source 1..22: mol_type = other DNA
gccacactgttccaatctatga

SEQ ID NO: 37 - DNA, 22 bp, synthetic construct
  misc_feature 1..22: note = primer Ta_Actin_R
  source 1..22: mol_type = other DNA
gccacactgttccaatctatga

SEQ ID NO: 38 - DNA, 24 bp, synthetic construct
  misc_feature 1..24: note = primer WORF256_F
  source 1..24: mol_type = other DNA
atccccaagctctagctcatttag

SEQ ID NO: 39 - DNA, 22 bp, synthetic construct
  misc_feature 1..22: note = primer WORF256_R
  source 1..22: mol_type = other DNA
gggggctggaagagaaaagaat

SEQ ID NO: 40 - protein, 22 aa, synthetic construct
  REGION 1..22: note = FLAG
DYKDHDGDYKDHDIDYKDDDDK

SEQ ID NO: 41 - RNA, 18 nt, Triticum timopheevii
  source 1..18: mol_type = transcribed RNA
cacctgtgcacttattta

SEQ ID NO: 42 - RNA, 18 nt, Triticum timopheevii
  source 1..18: mol_type = transcribed RNA
tctatctgagcctttacg

SEQ ID NO: 43 - RNA, 20 nt, Triticum timopheevii
  source 1..20: mol_type = transcribed RNA
cacctgtgcacttatttatg

SEQ ID NO: 44 - protein, 788 aa, synthetic construct
  REGION 1..788: note = Optimized RFL29a 1
MPRFSSTTPMSPPRLRLRLCARHSSSTSHPSRIWDPHAAFAAAAQRASSGTLTTEDAHHLFDELLRRGNPVQERPLNKFLAALARAPASASCCDGPALAVALFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAILLHGYAAEGCLVDMINLFNSMERDCILPDCRIFNILINAYAKSGKLDKAMLIFNEMQKQGVSPNAVTYSTVIHAFCKKGRLDDAVIKFNQMIDTGVRPNASVYRPLIQGFCTHGDLVKAKEYVTEMMKKGMPPPDIMFFNSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 45 - protein, 788 aa, synthetic construct
  REGION 1..788: note = Optimized RFL29a 2
MPRFSSTTPMSPPRLRLRLCARHSSSTSHPSRIWDPHAAFAAAAQRASSGTLTTEDAHHLFDELLRRGNPVQERPLNKFLAALARAPASASCCDGPALAVALFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAILLHGYAAEGCLVDMINLFNSMERDCILPDCRIFNILINAYAKSGKLDKAMLIFNEMQKQGVSPNAVTYSTVIHAFCKKGRLDDAVIKFNQMIDTGVRPNASVYNPLIQGFCTHGDLVKAKEYVTEMMKKGMPPPDIMFFNSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 46 - DNA, 2367 bp, synthetic construct
  misc_feature 1..2367: note = Optimized RFL29a 3
  source 1..2367: mol_type = other DNA
atgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccgaacgcatctgtttatcgtcccctaatccagggtttttgtacacatggcgatttggtgaaagcaaaggaatatgttactgaaatgatgaagaaaggtatgcctcctcctgatattatgttcttcaattcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 47 - DNA, 2367 bp, synthetic construct
  misc_feature 1..2367: note = Optimized RFL29a 4
  source 1..2367: mol_type = other DNA
atgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccgaacgcatctgtttataatcccctaatccagggtttttgtacacatggcgatttggtgaaagcaaaggaatatgttactgaaatgatgaagaaaggtatgcctcctcctgatattatgttcttcaattcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 48 - DNA, 2000 bp, Triticum aestivum
  source 1..2000: mol_type = genomic DNA
tttcggcggacgatgctctccgcaggttgcactggactagcttggtgtgaggcttgcaactttctcctgtgatgctcatcaacaaaatcggagatgtcggtcctcgacgcctgcggccgatcatttggcttggctgtcgaggtcctcgggtgccgtttgttcggaggggtcttgatgggtcgtcagcgaggaaatcggagttgcctgctcgtggagaggcgatgacgatgacattgcagcctcgacggtgtcctctcgtcagcagtatgtgtgtgatcttgtcagtgccaggtcggctggagtgccctattcgatgggcgtgttttaattaaccgggcaactctcttcttcttaatcagtgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccatgccacatcgatagagggtctaaccgccaggcgagaagcttcgcaaagagacccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtacctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttaggggacgcgcgcgccgagagagagagcagatttggggaagaaataaagccagggactggggaatgagaagataatgtttataaggggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggcc

SEQ ID NO: 49 - DNA, 812 bp, Triticum aestivum
  source 1..812: mol_type = genomic DNA
ttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtccctgctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcgtacatgcttgtctag

SEQ ID NO: 50 - DNA, 5179 bp, synthetic construct
  misc_feature 1..5179: note = optimised RFL29a expression cassette 1
  source 1..5179: mol_type = other DNA
tttcggcggacgatgctctccgcaggttgcactggactagcttggtgtgaggcttgcaactttctcctgtgatgctcatcaacaaaatcggagatgtcggtcctcgacgcctgcggccgatcatttggcttggctgtcgaggtcctcgggtgccgtttgttcggaggggtcttgatgggtcgtcagcgaggaaatcggagttgcctgctcgtggagaggcgatgacgatgacattgcagcctcgacggtgtcctctcgtcagcagtatgtgtgtgatcttgtcagtgccaggtcggctggagtgccctattcgatgggcgtgttttaattaaccgggcaactctcttcttcttaatcagtgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccatgccacatcgatagagggtctaaccgccaggcgagaagcttcgcaaagagacccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtacctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttaggggacgcgcgcgccgagagagagagcagatttggggaagaaataaagccagggactggggaatgagaagataatgtttataaggggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccgaacgcatctgtttatcgtcccctaatccagggtttttgtacacatggcgatttggtgaaagcaaaggaatatgttactgaaatgatgaagaaaggtatgcctcctcctgatattatgttcttcaattcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtccctgctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcgtacatgcttgtctag

SEQ ID NO: 51 - DNA, 5179 bp, synthetic construct
  misc_feature 1..5179: note = optimised RFL29a expression cassette 2
  source 1..5179: mol_type = other DNA
tttcggcggacgatgctctccgcaggttgcactggactagcttggtgtgaggcttgcaactttctcctgtgatgctcatcaacaaaatcggagatgtcggtcctcgacgcctgcggccgatcatttggcttggctgtcgaggtcctcgggtgccgtttgttcggaggggtcttgatgggtcgtcagcgaggaaatcggagttgcctgctcgtggagaggcgatgacgatgacattgcagcctcgacggtgtcctctcgtcagcagtatgtgtgtgatcttgtcagtgccaggtcggctggagtgccctattcgatgggcgtgttttaattaaccgggcaactctcttcttcttaatcagtgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccatgccacatcgatagagggtctaaccgccaggcgagaagcttcgcaaagagacccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtacctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttaggggacgcgcgcgccgagagagagagcagatttggggaagaaataaagccagggactggggaatgagaagataatgtttataaggggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccgaacgcatctgtttataatcccctaatccagggtttttgtacacatggcgatttggtgaaagcaaaggaatatgttactgaaatgatgaagaaaggtatgcctcctcctgatattatgttcttcaattcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatagttggtacatgatatctgaaatttaatttgcatcgcttgccatgggtccgtctgcttgtacaagaaatgcattttctatttgtaaataggaagtcagtttagaacaagccatcaggatgacgcaaagagtacaattcagttgcaccaccaataaaaaggcagaactagggctgccaaaacaacactgaatcaaaactcaaacagaaggagcagcaaacttttttttttttgaagctggacagtctgctagccaaacaactacaggagactgtcaggcggggcatgtagtggctggcgtctaagcgcctttgcttcttccaccatccatggcttagcctcacacggaatcgagtcaaccaattcccgtcggttttgggtggctcccttgaagatgcaattgttttcagcggccagatacgcatggtcagattaatcagcgagtgtgccccttctgtctggttcgagaaagaatttgaagagctgagcttgtccctgctggaaccagtttgaggttagttaatcataacagggtactagagaggtgttttattgactgttgatgtgtaatatgttatatgccatcctcttgatgattacggtgatctgtgaagaggcagcatgcaaaagtctgaatcacatatgcttatgtaattgtgttattatttgtgcagcttctagaccttttgccttgtagatggctacatggatctagttgtagcaatctgtaactgttagtgttttgtatatgctggcattgatgattagggtgacttgtgaagcatgcagaagtctgaatcgtacatgcttgtctag

SEQ ID NO: 52 - DNA, 22 bp, synthetic construct
  misc_feature 1..22: note = Primer KASPar AS1
  source 1..22: mol_type = other DNA
agaggcaaattgggcaaaggac

SEQ ID NO: 53 - DNA, 25 bp, synthetic construct
  misc_feature 1..25: note = Primer KASPar AS2
  source 1..25: mol_type = other DNA
agaatctgtctctccattcctcgtg

SEQ ID NO: 54 - DNA, 26 bp, synthetic construct
  misc_feature 1..26: note = Primer KASPar C
  source 1..26: mol_type = other DNA
atggtgtaagaccgtcgactatttgg